(19) United States
(12) Patent Application Publication
(10) Pub. No.: US 2020/0407434 A1
(43) Pub. Date: Dec. 31, 2020

(54) BISPECIFIC ANTIBODIES TO MOSPD2 AND T CELL- OR NK CELL-SPECIFIC MOLECULES

(71) Applicant: Vascular Biogenics Ltd., Modiin (IL)

(72) Inventors: Itzhak MENDEL, Rehovot (IL); Yaniv SALEM, Kyriat Ono (IL); Eyal BREITBART, Hashmonaim (IL)

(21) Appl. No.: 16/980,659

(22) PCT Filed: Mar. 13, 2019
(86) PCT No.: PCT/IB2019/052049

§ 371 Date: Sep. 14, 2020

Related U.S. Application Data
(60) Provisional application No. 62/642,339, filed on Mar. 13, 2018.

(51) Int. Cl. C07K 16/18; C07K 16/28; A61K 39/395; A61K 45/06; G01N 33/574

(52) U.S. Cl. CPC C07K 16/18; A61K 2039/505; A61K 39/39558; A61K 39/39541; A61K 45/06; G01N 33/57492; C07K 2317/31; C07K 2317/54; C07K 2317/92; C07K 2317/34; C07K 2317/73; C07K 2317/74; C07K 2317/21; C07K 2317/24; C07K 2317/622; C07K 16/2809

(57) ABSTRACT

Disclosed herein are bispecific antibodies or antigen binding fragments thereof that specifically bind to Motile Sperm Domain Containing Protein 2 (MOSPD2) and to a T cell-specific or NK cell-specific receptor molecule, pharmaceutical compositions and kits containing the same, and methods of making and using the same.

### REFERENCE TO SEQUENCE LISTING SUBMITTED ELECTRONICALLY

The content of the electronically submitted sequence listing (Name: 3182089PC01_SequenceListing_ST25.txt; Size: 34,597 bytes; and Date of Creation: Mar. 12, 2019, filed herewith, is incorporated herein by reference in its entirety.

### FIELD OF THE INVENTION

The present invention relates to bispecific antibodies or antigen binding fragments thereof that specifically bind to Motile Sperm Domain Containing Protein 2 (MOSPD2) and to T cell-specific or NK cell-specific receptor molecules, pharmaceutical compositions and kits containing the same, and methods of making and using the same.

### BACKGROUND OF THE INVENTION

MOSPD2 is a 518-amino acid long, highly conserved protein with 90% homology between human and mouse. Bioinformatic analyses indicate that MOSPD2 contains a CRAL-TRIO region, named after the cellular retinaldehyde-binding protein (CRALBP) and the TRIO protein. MOSPD2 also contains a structurally related region to the nematode major sperm protein and one transmembrane region.

MOSPD2 is expressed on monocytes that have infiltrated into inflamed tissues and on a variety of tumor types (Int'l Pub. No. WO 2017/021857). It is associated with metastasis of cancer cells and promotes monocyte migration (Int'l Pub. No. WO 2017/021857). Accordingly, inhibition of MOSPD2 (e.g., with anti-MOSPD2 antibodies) has been described as a treatment for inflammatory diseases and disorders (Int'l Pub. No. WO 2017/021855) and for cancer and cancer metastasis (Int'l Pub. No. WO 2017/021857).

Antibodies have been used to activate T cells to recognize and attack tumors. For example, antibodies against the antigen-specific T cell receptor (TCR)/CD3 complex have been reported to activate T cells. Davis et al., J. Immunol. 137:3758-3767 (1986). Furthermore, bispecific antibodies which recognize both an antigen on tumor cells and the TCR/CD3 complex have been reported to activate T cells and cause T cell-mediated lysis of tumor cells. Jung et al., Proc. Natl. Acad. Sci. U.S.A. 84:4611-4615 (1987); Jung et al., Immunol. Today 9:257-260 (1988); Staerz et al., Nature 314:628-631 (1985); and Perez et al., Nature 316:354-356 (1985). Bispecific antibodies to MOSPD2 are needed.

### SUMMARY OF THE INVENTION

In some embodiments, the present invention relates to a bispecific antibody or antigen binding fragment thereof comprising (i) one or more antigen binding domains to Motile Sperm Domain Containing Protein 2 (MOSPD2), and (ii) one or more antigen binding domains to a T cell- or NK cell-specific receptor molecule. In some embodiments, the T cell- or NK cell-specific receptor molecule is CD3, the T cell receptor (TCR), CD28, CD16, NKG2D, Ox40, 4-1BB, CD2, CD5, or CD95. In some embodiments, the one or more antigen binding domains to MOSPD2 is a Fab, Fab′, F(ab′)₂, Fv, scFv, sdFv fragment, heavy chain variable region, light chain variable region, complementarity determining region (CDR), heavy chain CDR1, heavy chain CDR2, heavy chain CDR3, light chain CDR1, light chain CDR2, or light chain CDR3 of an anti-MOSPD2 antibody or antigen binding fragment thereof. In some embodiments, the T cell- or NK cell-specific receptor molecule is CD3 and the one or more antigen binding domains to CD3 is a Fab, Fab′, F(ab′)₂, Fv, scFv, sdFv fragment, heavy chain variable region, light chain variable region, CDR, heavy chain CDR1, heavy chain CDR2, heavy chain CDR3, light chain CDR1, light chain CDR2, or light chain CDR3 of an anti-CD3 antibody or antigen binding fragment thereof.

In some embodiments, the bispecific antibody or antigen binding fragment thereof comprises one or more of the following antigen binding domains:

(i) a heavy chain variable region of an anti-MOSPD2 antibody or antigen binding fragment thereof; and

(ii) a light chain variable region of an anti-MOSPD2 antibody or antigen binding fragment thereof.

In some embodiments, the bispecific antibody or antigen binding fragment thereof comprises one or more of the following antigen binding domains:

(i) a heavy chain variable region of an anti-CD3 antibody or antigen binding fragment thereof; and

(ii) a light chain variable region of an anti-CD3 antibody or antigen binding fragment thereof.

In some embodiments, the bispecific antibody or antigen binding fragment thereof comprises the following antigen binding domains:

(i) a heavy chain variable region of an anti-MOSPD2 antibody or antigen binding fragment thereof;

(ii) a light chain variable region of an anti-MOSPD2 antibody or antigen binding fragment thereof;

(iii) a heavy chain variable region of an anti-CD3 antibody or antigen binding fragment thereof; and

(iv) a light chain variable region of an anti-CD3 antibody or antigen binding fragment thereof.

In some embodiments, the bispecific antibody or antigen binding fragment thereof comprises the following antigen binding domains in order from the N-terminus to the C-terminus:

(i) a heavy chain variable region of an anti-MOSPD2 antibody or antigen binding fragment thereof;

(ii) a light chain variable region of an anti-MOSPD2 antibody or antigen binding fragment thereof;

(iii) a heavy chain variable region of an anti-CD3 antibody or antigen binding fragment thereof; and

(iv) a light chain variable region of an anti-CD3 antibody or antigen binding fragment thereof.

In some embodiments, one or more of the antigen binding domains of the bispecific antibody or antigen binding fragment thereof are joined by a peptide linker. In some embodiments, one or more of the antigen binding domains of the bispecific antibody or antigen binding fragment thereof are joined by peptide linkers. In some embodiments, all of the antigen binding domains of the bispecific antibody or antigen binding fragment thereof are joined by peptide linkers.

In some embodiments, at least one antigen binding domain of the bispecific antibody or antigen binding fragment thereof is human or humanized.

In some embodiments, the bispecific antibody or antigen binding fragment thereof is a single-chain polypeptide.

In some embodiments, the bispecific antibody or antigen binding fragment thereof has a molecular weight of no more than about 60,000 Daltons.

In some embodiments, the bispecific antibody is a nanobody, diabody, CrossMab, duobody, bivalent antibody, bispecific T cell engager (BiTE), dual affinity retargeting (DART), triple body, miniantibody, TriBi minibody, intrabody, or quadroma.

In some embodiments, the bispecific antibody or antigen binding fragment thereof specifically binds to MOSPD2 and/or CD3 with an equilibrium dissociation constant (K_{D}) of from about 10⁻⁶M to about 10⁻¹²M.

In some embodiments, the bispecific antibody or antigen binding fragment, or one or more antigen binding domains to MOSPD2, specifically binds to one or more of the following amino acid regions of MOSPD2, numbered according to SEQ ID NO:1: about 505 to about 515, about 500 to about 515, about 230 to about 240, about 510 to about 520, about 210 to about 220, about 15 to about 25, about 505 to about 520, about 505 to about 515, about 90 to about 100, about 505 to about 525, about 230 to about 245, about 505 to about 510, about 130 to about 140, about 220 to about 230, about 15 to about 30, about 80 to about 95, about 40 to about 50, about 460 to about 475, about 340 to about 350, about 500 to about 515, about 460 to about 470, about 325 to about 335, about 20 to about 35, about 215 to about 225, about 510 to about 520, about 175 to about 190, about 500 to about 510, about 505 to about 530, about 60 to about 75, about 500 to about 520, about 145 to about 160, about 502 to about 515, about 85 to about 100, about 205 to about 220, about 175 to about 190, about 500 to about 505, about 500 to about 525, about 495 to about 505, about 495 to about 510, about 190 to about 200, about 190 to about 198, about 502 to about 515, about 1 to about 60, about 80 to about 240, about 90 to about 235, about 330 to about 445, about 330 to about 430, about 495 to about 515, about 145 to about 240, about 145 to about 220, about 145 to about 200, about 160 to about 240, about 160 to about 220, about 160 to about 200, about 175 to about 240, about 175 to about 220, about 175 to about 200, about 170 to about 190, about 178 to about 185, about 85 to about 140, about 85 to about 130, about 90 to about 140, about 90 to about 130, about 95 to about 140, about 95 to about 130, about 100 to about 130, about 100 to about 140, about 110 to about 130, or about 115 to about 127.

In some embodiments, the bispecific antibody or antigen binding fragment, or one or more antigen binding domains to MOSPD2, specifically binds to one or more of SEQ ID NOs:1-4, or a functional variant thereof, or to a polypeptide encoded by one or more of SEQ ID NOs:5-8, or a functional variant thereof.

In some embodiments, the bispecific antibody or antigen binding fragment, or one or more antigen binding domains to MOSPD2, specifically binds to MOSPD2 with a K_{D} of from about 10⁻⁶ M to about 10⁻¹² M.

In some embodiments, the present invention relates to a nucleic acid encoding a bispecific antibody or antigen binding fragment thereof described herein.

In some embodiments, the present invention relates to an expression vector comprising a nucleic acid encoding a bispecific antibody or antigen binding fragment thereof described herein.

In some embodiments, the present invention relates to a pharmaceutical composition comprising a bispecific antibody or antigen binding fragment thereof described herein and a pharmaceutically acceptable carrier. In some embodiments, the pharmaceutical composition is suitable for systemic or local administration. In some embodiments, the pharmaceutical composition is suitable for nasal, oral, intra-peritoneal, or intra-tumor administration. In some embodiments, the pharmaceutical composition is suitable for intravenous administration, intramuscular administration, or subcutaneous administration.

In some embodiments, the present invention relates to a method of treating or preventing cancer in a subject comprising administering to the subject a bispecific antibody or antigen binding fragment thereof described herein or a pharmaceutical composition described herein in an effective amount to treat or prevent cancer.

In some embodiments, the present invention relates to a method of treating or preventing cancer metastasis in a subject comprising administering to the subject a bispecific antibody or antigen binding fragment thereof described herein or a pharmaceutical composition described herein in an effective amount to treat or prevent cancer metastasis.

In some embodiments, the method further comprises administering to the subject an effective amount of an anticancer drug. In some embodiments, the anticancer drug is Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and I 131 Iodine Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPDX, Carboplatin, Carboplatin-Taxol, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, Chlorambucil-Prednisone, CHOP, Cisplatin, Clafen (Cyclophosphamide), Clofarabine, CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), Folfiri, Folfiri-Bevacizumab, Folfiri-Cetuximab, Folfirinox, Folflox, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, OEPA, Ofatumumab, OFF, Olaparib, Omacetaxine Mepesuccinate, Oncaspar (Pegaspargase), Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and I 131 Iodine Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate), or a combination thereof.

In some embodiments, the present invention relates to a method for inhibiting or reducing tumor cells in a subject comprising administering to the subject a bispecific antibody or antigen binding fragment described herein or a pharmaceutical composition described herein. In some embodiments of the method, the number of tumor cells are reduced by at least about 10%, at least about 20%, at least about 30%, at least about 40%, at least about 50%, at least about 60%, at least about 70%, at least about 80%, at least about 90%, or at least about 100%, compared to a control or reference value.

In some embodiments, the present invention relates to a method of increasing the production of cytokines by cells expressing CD3 in a subject comprising administering to the subject a bispecific antibody or antigen binding fragment thereof described herein or a pharmaceutical composition described herein.

In some embodiments, the present invention relates to a method of increasing IL-2, CD69, and/or IFN-γ production or concentration in a T cell comprising contacting the T cell with a bispecific antibody or antigen binding fragment thereof described herein or a pharmaceutical composition described herein. In some embodiments, IFN-γ production increases by at least about 100%, at least about 200%, at least about 300%, at least about 400%, at least about 500%, at least about 600%, at least about 700%, at least about 800%, at least about 900%, or at least about 1000% compared to a control or reference value. In some embodiments, the IFN-γ concentration increases by at least about 1000 pg/ml, at least about 2000 pg/ml, at least about 3000 pg/ml, at least about 4000 pg/ml, at least about 5000 pg/ml, at least about 6000 pg/ml, at least about 7000 pg/ml, at least about 8000 pg/ml, at least about 9000 pg/ml, or at least about 10000 pg/ml compared to a control or reference value. In some embodiments, CD69 production increases by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, or at least about 30% compared to a control or reference value.

In some embodiments, the present invention relates to a method of stimulating an immune response in a subject comprising administering to the subject a bispecific antibody or antigen binding fragment thereof described herein or a pharmaceutical composition described herein.

In some embodiments, the present invention relates to a method of stimulating a T cell-mediated cytotoxic immune response against a cancer cell in a subject comprising administering to the subject a bispecific antibody or antigen binding fragment thereof described herein or a pharmaceutical composition described herein.

In some embodiments, the present invention relates to a method of increasing T cell proliferation comprising contacting a T cell with a bispecific antibody or antigen binding fragment thereof described herein or a pharmaceutical composition described herein.

In some embodiments, the present invention relates to a method of reducing or depleting the number of T regulatory cells in a tumor of a subject comprising administering to the subject a bispecific antibody or antigen binding fragment thereof described herein or a pharmaceutical composition described herein.

In some embodiments, the present invention relates to a method for the prediction, diagnosis, or prognosis of cancer or cancer metastasis in a subject comprising determining the expression level of MOSPD2 in a sample of the subject using a bispecific antibody or antigen binding fragment thereof described herein. In some embodiments, the method comprises (i) determining or quantifying the expression level of MOSPD2 in a sample of the subject using a bispecific antibody or antigen binding fragment thereof described herein, and (ii) comparing the expression level obtained in step (i) with a control or reference value, wherein an increased expression level of MOSPD2 with respect to the control or reference value is indicative of cancer, an increased risk of developing cancer, or a poor cancer prognosis.

In some embodiments, the present invention relates to a method for the prediction, diagnosis, or prognosis of tumor progression or tumor invasiveness in a subject comprising determining the expression level of MOSPD2 in a sample of the subject using a bispecific antibody or antigen binding fragment thereof described herein. In some embodiments, the method comprises (i) determining or quantifying the expression level of MOSPD2 in a sample of the subject using a bispecific antibody or antigen binding fragment thereof described herein, and (ii) comparing the expression level obtained in step (i) with a control or reference value, wherein an increased expression level of MOSPD2 with respect to the control or reference value is indicative or a poor tumor progression or tumor invasiveness prognosis.

In some embodiments, the method for prediction, diagnosis, or prognosis further comprises one or more of the following steps:

instructing a laboratory to quantify the expression level of MOSPD2 in the sample;

obtaining a report of the expression level of MOSPD2 in the sample from a laboratory; and/or

administering a therapeutically effective amount of an inhibitor of MOSPD2 (e.g., an anti-MOSPD2 antibody) to the subject.

In some embodiments, the sample in a method for prediction, diagnosis, or prognosis is a tissue biopsy, tumor biopsy, or blood sample from the subject.

In some embodiments, the control or reference value in a method for prediction, diagnosis, or prognosis is the expression level of MOSPD2 in normal tissue or normal adjacent tissue (NAT). In some embodiments, the control or reference value in a method for prediction, diagnosis, or prognosis is no detectable MOSPD2 expression or no significant MOSPD2 expression.

In some embodiments, the present invention relates to a method for treating or preventing a MOSPD2-expressing tumor in a subject comprising administering to the subject a bispecific antibody or antigen binding fragment thereof described herein or a pharmaceutical composition described herein in an effective amount to treat or prevent a MOSPD2 expressing tumor.

In some embodiments, the present invention relates to a method for treating or preventing a tumor having MOSPD2-expressing tumor associated macrophages in a subject comprising administering to the subject a bispecific antibody or antigen binding fragment thereof described herein or a pharmaceutical composition described herein in an effective amount to treat or prevent a tumor having MOSPD2-expressing tumor associated macrophages.

In some embodiments, the present invention relates to a method for producing a bispecific antibody or antigen binding fragment thereof described herein comprising (i) culturing a host cell transformed with a nucleic acid described herein or an expression vector described herein, and (ii) collecting and purifying the expressed bispecific antibody or antigen binding fragment thereof.

In some embodiments, the present invention relates to a kit comprising (i) a bispecific antibody or antigen binding fragment thereof described herein or a pharmaceutical composition described herein, and (ii) instructions for use.

### BRIEF DESCRIPTION OF THE DRAWINGS

Some embodiments of the invention are described herein, by way of example only, with reference to the accompanying drawings. With specific reference now to the drawings in detail, it is stressed that the particulars shown are by way of example and for purposes of illustrative discussion of embodiments of the invention.

FIG. 1 shows that MOSPD2 promotes migration of metastatic breast cancer and melanoma cells. MOSPD2 expression in MDA-231 breast cancer and A2058 melanoma cells was silenced by transducing the cells with sh-MOSPD2 lenti-virus particles. Western blots in FIG. 1 show cells transduced with sh-MOSPD2 have decreased MOSPD2 protein expression. FIG. 1 also shows migration test results of cells transduced with sh-control or sh-MOSPD2 lenti-virus particles in a trans-well migration assay towards 10% fetal calf serum (FCS) and EGF (200 ng/ml).

FIG. 2 shows that silencing of MOSPD2 does not affect cell viability or proliferation of MDA-231 cells. MDA-231 cells transduced with sh-control or sh-MOSPD2 lenti-virus particles were seeded, collected and counted every 24 hours for three consecutive days. Cell proliferation rates were measured and expressed in FIG. 2 as mean±standard deviation of triplicates.

FIGS. 3A-3C show in vivo test results of metastasis of MDA-231 cells with or without MOSPD2 silencing. In FIG. 3A, MDA-231 cells transduced with sh-control or sh-MOSPD2 lenti-virus particles were injected (10⁶) in the tail vein of SCID mice (n=10/group). Mice were sacrificed on day 28, their lungs were harvested for H&E staining, and tumor area was determined. The results shown in FIG. 3A are expressed as mean±standard error of measured metastasis size (*p<0.05).

In FIGS. 3B and 3C, MDA-231 cells transduced with sh-control or sh-MOSPD2 lenti-virus particles (n=13 and n=8, respectively) were injected (5×10⁶) in the mammary fat pad of SCID mice. Mice were sacrificed on day 56. The ipsilateral inguinal lymph node was excised (FIG. 3B), their lungs were harvested for H&E staining, and tumor area was determined (FIG. 3C). The results shown in FIG. 3C are expressed as mean±standard error of measured metastasis size. The tumor area for sh-control transduced cells is 1376.9±752.6 (n=13), compared to 550.0±326.2 (n=8) for sh-MOSPD2 transduced cells.

FIGS. 3A-3C show that MOSPD2 promotes metastasis of MDA-231 breast cancer cells in vivo.

FIGS. 4A-4E show a comparison of MOSPD2 expression levels in various human cancer tissues to those of their respective normal tissue counterparts. Slides containing various normal and cancerous human tissues were stained with control or anti-MOSPD2 antibody. Cancer tissues that stained positively for MOSPD2 are shown. FIGS. 4A-4E show that MOSPD2 is expressed in various human cancer tissues.

FIGS. 5A and 5B show the results of cancer cells transduced with control or MOSPD2 CRISPR-CAS9 lenti-virus particles tested in a trans-well migration assay. Cells were seeded in the upper compartment and attracted to the lower compartment using medium supplemented with 10% FCS and EGF (200 ng/ml). The graph shown in FIG. 5A was determined by fluorescence-activated cell sorting (FACS) with results expressed as mean±standard deviation of triplicates. The images shown in FIG. 5B are from visual recordation. In FIGS. 5A and 5B, MDA-231 cells were transduced with lenti-viral particles with plasmids containing a control or MOSPD2 CRISPR-CAS9 system. Western blots show decreased MOSPD2 protein expression in cells transduced with the MOSPD2 CRISPR-CAS9 system (inset). FIGS. 5A and 5B show that CRISPR-CAS9 driven MOSPD2 gene editing inhibits breast cancer cell migration.

FIG. 5C shows the effect of MOSPD2 silencing on phosphorylation events associated with cell migration. MDA-231 transduced with control or MOSPD2 CRISPR-CAS9 lenti-virus particles were incubated with 10% FCS and EGF (400 ng/ml) for 10 minutes. Phosphorylation of ERK, AKT and FAK was determined using Western blot. HSP90 was used as a loading control. FIG. 5C shows that MOSPD2 silencing by CRISPR-CAS9 driven gene editing inhibits phosphorylation events associated with cell migration.

FIG. 5D shows in vivo test results of metastasis of MDA-231 breast cancer cells transduced with control or MOSPD2 CRISPR-CAS9 lenti-virus particles. In FIG. 5D, 10⁶ control or MOSPD2 CRISPR-CAS9 lenti-virus transduced MDA-231 cells were injected into the tail vein of 8 weeks old female SCID mice (C.B-17/IcrHsd-Prkdc^{scid}, Harlan Israel). Mice were sacrificed after 3 weeks and their lungs were excised for histopathologic examination. FIG. 5D shows that silencing of MOSPD2 by the CRISPR-CAS9 system significantly inhibits the presence of metastatic breast cancer cells in the lungs by more than 95% (metastasis area). P=0.002.

FIG. 6A-6B show anti-MOSPD2 F(ab′)₂ mAb binds to MOSPD2 on A2058 melanoma and HepG2 liver cancer cell lines.

FIG. 7 shows anti-MOSPD2 F(ab′)₂ mAb significantly inhibited EGF-induced trans-well migration of MDA-231 cells.

FIGS. 8A-8F show histological images of human breast cancer samples from different pathological stages or from normal tissue adjacent to a tumor (normal adjacent tissue; NAT). The samples were mounted to slides and stained with anti-MOSPD2 antibody. FIGS. 8A-8F show that MOSPD2 expression was associated with the transition of breast cancer cells from locally-restricted tumors to invasive and metastatic tumors.

FIG. 9 shows scoring of MOSPD2 expression intensity (in a scale of 0-3, where 0 is no expression and 3 is very high expression) in samples from different stages of breast cancer or normal adjacent tissue (NAT) (*p<0.001).

FIGS. 10A-10D show images comparing MOSPD2 expression level in various normal and cancerous human tissues collected from the colon (FIGS. 10A-10B) or the liver (FIGS. 10C-10D). MOSPD2 was expressed in 67% of colon adenocarcinoma and in 45% of hepatocellular carcinoma samples, while no expression was detected in normal colon and liver tissues.

FIGS. 11A-11E show a comparison of MOSPD2 expression level in normal tissue, NAT and cancerous tissue at different grades collected from human liver. Samples were mounted to slides and stained with anti-MOSPD2 antibody. FIGS. 11C-11E show that MOSPD2 staining intensity increased as tumor grade of hepatocellular carcinoma increased.

FIGS. 12A-12B show MOSPD2 expression intensity in samples collected from hepatocellular carcinoma. Samples were mounted to slides and stained with anti-MOSPD2 antibody. FIG. 12A shows that MOSPD2 expression was significantly increased (p<0.001) in samples collected from malignant hepatocellular carcinoma, compared to normal and NAT samples. FIG. 12B shows that MOSPD2 staining intensity increased significantly in correlation with the progression of hepatocellular carcinoma.

FIG. 13A shows that administration of increasing concentrations of anti-MOSPD2/anti-CD3 bispecific antibody (BiTE) results in a dose-dependent decrease in survival of solid tumor-derived cell lines and increase in T cell activation. *=P<0.01. FIGS. 13B and 13C show that administration of increasing concentrations of BiTE also results in a dose-dependent increase in IFN-γ release from CD8 effector T cells added to HELA and A2058 cultures, respectively. N.D=not detectable.

FIG. 14A shows that administration of anti-MOSPD2/anti-CD3 bispecific antibody (BiTE) results in decreased survival of monocytic cell lines and increased T cell activation. **=P<0.001. FIG. 14B shows staining for CD3 and CD69 of T cells added to THP-1 and U937 cultures. FIG. 14C shows that administration of BiTE also results in an increase in IFN-γ release from T cells added to THP-1 and U937 cultures. N.D=not detectable.

FIG. 15 shows that administration of anti-MOSPD2/anti-CD3 bispecific antibodies results in a significant decrease in survival of MDA-231 breast cancer cells. Pre-activated CD8+ T cells were co-incubated with MDA-231 cells at the ratio of 5:1 respectively, and control antibody (IgG) or anti-MOSPD2/anti-CD3 bispecific antibodies (Bispecific Abl-Bispecific Ab4). After incubation for 24 hours, cells were collected and counted using FACScalibur. Results shown are mean cell counts for each treatment±standard error from triplicate wells. **p<0.005; ***p<0.001.

### DETAILED DESCRIPTION OF THE INVENTION

Before explaining embodiments of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

### General Definitions

The terms “comprises”, “comprising”, “includes”, “including”, “having”, and their conjugates mean “including but not limited to.”

The term “consisting of” means “including and limited to.”

The term “consisting essentially of” means the specified material of a composition, or the specified steps of a method, and those additional materials or steps that do not materially affect the basic characteristics of the material or method.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention can include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. Whether or not modified by the term “about”, the claims include equivalents of the recited quantities. In one embodiment, the term “about” means within 10% of the reported numerical value. In another embodiment, the term “about” means within 5% of the reported numerical value.

Throughout this application, various embodiments of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5 and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, “MOSPD2” refers to any polypeptide classified as a Motile Sperm Domain Containing Protein 2 or having equivalent function. Examples of MOSPD2 include, but are not limited to, a polypeptide of any one of SEQ ID NOs:1-4, or a functional variant thereof (e.g., having a sequence at least 75%, at least 80%, at least 85%, at least 90%, at least 95%, at least 96%, at least 97%, at least 98%, or at least 99% identical to any one of SEQ ID NOs:1-4). Other examples of MOSPD2 include, but are not limited to, a polypeptide encoded by a polynucleotide of any one of SEQ ID NOs:5-8, or a functional variant thereof (e.g., a polynucleotide having at least 75%, at least 80%, at least 85%, at least 90%, at least 95%, at least 96%, at least 97%, at least 98%, or at least 99% identical to any one of SEQ ID NOs:5-8). Other examples of MOSPD2 include the lipid-associated molecules (“LIPAMs”) disclosed in U.S. Appl. Pub. No. 2004/0171009 which is incorporated herein by reference in its entirety. LIPAM-5 is 100% identical to SEQ ID NO:1, except that amino acid residue 482 is serine instead of arginine. Other examples of MOSPD2 can be identified by searching public databases (e.g., BLAST), as well known to one skilled in the art.

In any of the embodiments described herein, MOSPD2 can be MOSPD2 expressed by a cancer cell, e.g., a human cancer cell. Also, in any of the embodiments described herein, MOSPD2 can be a mammalian MOSPD2 or a human MOSPD2.

An “antibody” or an “antigen binding fragment” of an antibody include, but are not limited to, polyclonal, monoclonal, murine, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), a light chain variable region (VL) or a heavy chain variable region (VH) domain, fragments comprising either a VL or VH domain, and fragments produced by a Fab expression library. An antibody or antigen binding fragment of an antibody can be of any type (e.g., IgG, IgE, IgM, IgD, IgA, and IgY), class (e.g., IgG1, IgG2, IgG3, IgG4, IgA1 and IgA2) or subclass of immunoglobulin molecule. Methods for making an antigen binding fragment of an antibody are known and include, for example, chemical or protease digestion of an antibody.

As used herein, an “antigen binding domain” is a polypeptide region of an antibody or fragment thereof that specifically binds to an antigen. Examples of an antigen binding domain include, but are not limited to, Fab, Fab′, F(ab′)₂, Fv, scFv, sdFv fragment, heavy chain variable region, light chain variable region, complementarity determining region (CDR), heavy chain CDR1, heavy chain CDR2, heavy chain CDR3, light chain CDR1, light chain CDR2, or light chain CDR3.

As used herein, a “T cell- or NK cell-specific receptor molecule” refers to any molecule, compound or peptide that specifically binds to, or directs another molecule, compound or peptide to specifically bind to, a T cell-specific receptor or NK cell-specific receptor. Examples include, but are not limited to, CD3, the T cell receptor (TCR), CD28, CD16, NKG2D, Ox40, 4-1BB, CD2, CD5, and CD95.

By “specifically binds,” it is generally meant that an antibody or fragment, variant, or derivative thereof binds to an epitope by its antigen binding domain, and that the binding entails some complementarity between the antigen binding domain and the epitope. According to this definition, an antibody or fragment, variant, or derivative thereof is said to “specifically bind” to an epitope when it binds to that epitope via its antigen binding domain more readily than it would bind to a random, unrelated epitope.

As used herein, an “epitope” refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by methods described in the literature and herein, e.g., NMR spectroscopy, X-ray diffraction crystallography studies, ELISA assays, hydrogen/deuterium exchange coupled with mass spectrometry (e.g., MALDI mass spectrometry), array-based oligo-peptide scanning assays, and/or mutagenesis mapping (e.g., site-directed mutagenesis mapping).

The term “percent identity,” as known in the art, is a relationship between two or more polypeptide sequences or two or more polynucleotide sequences, as determined by comparing the sequences. In the art, “identity” and “sequence identity” also mean the degree of sequence relatedness between polypeptide or polynucleotide sequences, as the case may be, as determined by the match between strings of such sequences. “Identity” and “similarity” can be readily calculated by known methods and publicly available resources, including but not limited to those described in: (1) Computational Molecular Biology (Lesk, A. M., Ed.) Oxford University: NY (1988); (2) Biocomputing: Informatics and Genome Projects (Smith, D. W., Ed.) Academic: NY (1993); (3) Computer Analysis of Sequence Data, Part I (Griffin, A. M., and Griffin, H. G., Eds.) Humania: NJ (1994); (4) Sequence Analysis in Molecular Biology (von Heinje, G., Ed.) Academic (1987); and (5) Sequence Analysis Primer (Gribskov, M. and Devereux, J., Eds.) Stockton: NY (1991).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

### Bispecific Antibodies and Antigen Binding Fragments Thereof

In some embodiments, the present invention is related to a bispecific antibody or antigen binding fragment thereof that specifically binds to MOSPD2 and to a T cell- or NK cell-specific receptor molecule. In some embodiments, the bispecific antibody or antigen binding fragment thereof comprises (i) one or more antigen binding domains to MOSPD2, and (ii) one or more antigen binding domains to a T cell- or NK cell-specific receptor molecule.

In some embodiments, MOSPD2 is a polypeptide of any one of SEQ ID NOs:1-4, or a functional variant thereof (e.g., having a sequence at least 75%, at least 80%, at least 85%, at least 90%, at least 95%, at least 96%, at least 97%, at least 98%, or at least 99% identical to any one of SEQ ID NOs:1-4). In other embodiments, MOSPD2 is a polypeptide encoded by a polynucleotide of any one of SEQ ID NOs:5-8, or a functional variant thereof (e.g., a polynucleotide having a sequence at least 75%, at least 80%, at least 85%, at least 90%, at least 95%, at least 96%, at least 97%, at least 98%, or at least 99% identical to any one of SEQ ID NOs:5-8). In other embodiments, MOSPD2 is a lipid-associated molecule (“LIPAM”) disclosed in U.S. Appl. Pub. No. 2004/0171009, which is incorporated herein by reference in its entirety. LIPAM-5 is 100% identical to SEQ ID NO:1, except that amino acid residue 482 is serine instead of arginine. Other exemplary MOSPD2 can be identified by searching public databases (e.g., BLAST), as well known to one skilled in the art.

In some embodiments, the T cell- or NK cell-specific receptor molecule is CD3, the T cell receptor (TCR), CD28, CD16, NKG2D, Ox40, 4-1BB, CD2, CD5, or CD95. Other exemplary T cell- or NK cell-specific receptor molecules can be identified by searching public databases (e.g., BLAST), as well known to one skilled in the art.

In some embodiments, the antigen binding domain to MOSPD2 and/or the T cell- or NK cell-specific receptor molecule is a Fab, Fab′, F(ab′)₂, Fv, scFv, sdFv fragment, heavy chain variable region, light chain variable region, complementarity determining region (CDR), heavy chain CDR1, heavy chain CDR2, heavy chain CDR3, light chain CDR1, light chain CDR2, or light chain CDR3 of an antibody.

In some embodiments, the bispecific antibody or antigen binding fragment thereof comprises one or more of the following antigen binding domains:

(i) a heavy chain variable region of an anti-MOSPD2 antibody or antigen binding fragment thereof; and

(ii) a light chain variable region of an anti-MOSPD2 antibody or antigen binding fragment thereof.

In some embodiments, the bispecific antibody or antigen binding fragment thereof comprises one or more of the following antigen binding domains:

(i) a heavy chain variable region of an antibody or antigen binding fragment thereof that specifically binds to a T cell- or NK cell-specific receptor molecule (e.g., an anti-CD3 antibody or antigen binding fragment thereof); and

(ii) a light chain variable region of an antibody or antigen binding fragment thereof that specifically binds to a T cell- or NK cell-specific receptor molecule (e.g., an anti-CD3 antibody or antigen binding fragment thereof).

In some embodiments, the bispecific antibody or antigen binding fragment thereof comprises the following antigen binding domains:

(i) a heavy chain variable region of an anti-MOSPD2 antibody or antigen binding fragment thereof;

(ii) a light chain variable region of an anti-MOSPD2 antibody or antigen binding fragment thereof;

(iii) a heavy chain variable region of an antibody or antigen binding fragment thereof that specifically binds to a T cell- or NK cell-specific receptor molecule (e.g., an anti-CD3 antibody or antigen binding fragment thereof); and

(iv) a light chain variable region of an antibody or antigen binding fragment thereof that specifically binds to a T cell- or NK cell-specific receptor molecule (e.g., an anti-CD3 antibody or antigen binding fragment thereof).

In some embodiments, the bispecific antibody or antigen binding fragment comprises the following antigen binding domains in order from the N-terminus to the C-terminus:

(i) a heavy chain variable region of an anti-MOSPD2 antibody or antigen binding fragment thereof;

(ii) a light chain variable region of an anti-MOSPD2 antibody or antigen binding fragment thereof;

(iii) a heavy chain variable region of an antibody or antigen binding fragment thereof that specifically binds to a T cell- or NK cell-specific receptor molecule (e.g., an anti-CD3 antibody or antigen binding fragment thereof); and

(iv) a light chain variable region of an antibody or antigen binding fragment thereof that specifically binds to a T cell- or NK cell-specific receptor molecule (e.g., an anti-CD3 antibody or antigen binding fragment thereof).

In some embodiments, the bispecific antibody or antigen binding fragment comprises the following antigen binding domains in order from the N-terminus to the C-terminus:

(i) a heavy chain variable region of an antibody or antigen binding fragment thereof that specifically binds to a T cell- or NK cell-specific receptor molecule (e.g., an anti-CD3 antibody or antigen binding fragment thereof);

(ii) a light chain variable region of an antibody or antigen binding fragment thereof that specifically binds to a T cell- or NK cell-specific receptor molecule (e.g., an anti-CD3 antibody or antigen binding fragment thereof);

(iii) a heavy chain variable region of an anti-MOSPD2 antibody or antigen binding fragment thereof; and

(iv) a light chain variable region of an anti-MOSPD2 antibody or antigen binding fragment thereof.

In some embodiments, one or more of the antigen binding domains of the bispecific antibody or antigen binding fragment thereof are joined by a peptide linker. In some embodiments, all of the antigen binding domains of the bispecific antibody or antigen binding fragment thereof are joined by a peptide linker. In some embodiments, the peptide linker is from about 5 amino acids to about 50 amino acids in length (e.g., from about 5 amino acids to about 40 amino acids, about 5 amino acids to about 30 amino acids, about 5 amino acids to about 20 amino acids, about 5 amino acids to about 10 amino acids, about 10 amino acids to about 50 amino acids, about 10 amino acids to about 40 amino acids, about 10 amino acids to about 30 amino acids, about 10 amino acids to about 20 amino acids, about 20 amino acids to about 50 amino acids, about 20 amino acids to about 40 amino acids, about 20 amino acids to about 30 amino acids, about 30 amino acids to about 50 amino acids, about 30 amino acids to about 40 amino acids, or about 40 amino acids to about 50 amino acids in length). In some embodiments, the peptide linker is about 5 amino acids, about 10 amino acids, about 20 amino acids, about 30 amino acids, about 40 amino acids, or about 50 amino acids in length. In some embodiments, the peptide linker is composed of serine (S) and glycine (G) residues or alanine (A) and glycine residues. In some embodiments, the peptide linker is (GGGGS)n, (SGGGG)n, or (GGGGGGGG)n, where n is an integer from about 1 to about 10.

In some embodiments, at least one antigen binding domain of the bispecific antibody or antigen binding fragment is human or humanized.

In some embodiments, the bispecific antibody or antigen binding fragment thereof is a single-chain polypeptide.

In some embodiments, the bispecific antibody or antigen binding fragment thereof has a molecular weight of no more than about 60,000 Daltons.

In some embodiments, the bispecific antibody is a nanobody, diabody, CrossMab, duobody, bivalent antibody, bispecific T cell engager (BiTE), dual affinity retargeting (DART), triple body, miniantibody, TriBi minibody, intrabody, or quadroma.

In some embodiments, the bispecific antibody or antigen binding fragment thereof, or one or more antigen binding domains to MOSPD2, specifically binds to one or more of SEQ ID NOs:1-4, or a functional variant thereof, or to a polypeptide encoded by one or more of SEQ ID NOs:5-8, or a functional variant thereof.

In some embodiments, the bispecific antibody or antigen binding fragment thereof, or one or more antigen binding domains to MOSPD2, specifically binds to MOSPD2 with an equilibrium dissociation constant (K_{D}) of from about 10⁻⁶ M to about 10⁻¹² M. In some embodiments, the bispecific antibody or antigen binding fragment thereof specifically binds to a T cell- or NK cell-specific receptor molecule (e.g., CD3) and/or CD3 with a K_{D} of from about 10⁻⁶ M to about 10⁻¹² M.

In some embodiments the bispecific antibody or antigen binding fragment thereof, or one or more antigen binding domains to MOSPD2, specifically binds to MOSPD2 with a K_{D} of from about 10⁻⁶ M to about 10⁻¹² M, or any range of values thereof (e.g., from about 10⁻⁷ M to about 10⁻¹² M, from about 10⁻⁸M to about 10⁻¹² M, from about 10⁻⁹M to about 10⁻¹² M, from about 10⁻¹⁰ M to about 10⁻¹² M, from about 10⁻¹¹M to about 10⁻¹² M, from about 10⁻⁶ M to about 10⁻¹¹M, from about 10⁻⁷ M to about 10⁻¹¹ M, from about 10⁻⁸ M to about 10⁻¹¹ M, from about 10⁻⁹ M to about 10⁻¹¹M, from about 10⁻¹⁰ M to about 10⁻¹¹ M, from about 10⁻⁶ M to about 10⁻¹⁰ M, from about 10⁻⁷ M to about 10⁻¹⁰ M, from about 10⁻⁸M to about 10⁻¹⁰ M, from about 10⁻⁹ M to about 10⁻¹⁰ M, from about 10⁻⁶ M to about 10⁻⁹M, from about 10⁻⁷ M to about 10⁻⁹M, from about 10⁻⁸M to about 10⁻⁹M, from about 10⁻⁶ M to about 10⁻⁸ M, or from about 10⁻⁷ M to about 10⁻⁸).

In some embodiments, the bispecific antibody or antigen binding fragment thereof, or one or more antigen binding domains to MOSPD2, specifically binds to MOSPD2 with a K_{D} of about 10⁻⁶M, about 10⁻⁷M, about 10⁻⁸M, about 10⁻⁹M, about 10⁻¹⁰ M, about 10⁻¹¹M, or about 10⁻¹² M.

In some embodiments, the bispecific antibody or antigen binding fragment thereof, or one or more antigen binding domains to MOSPD2, specifically binds to one or more epitopes on MOSPD2. In some embodiments, the K_{D} is determined by Scatchard analysis, surface plasmon resonance, in some embodiments, at 37° C.

In some embodiments, the bispecific antibody or antigen binding fragment thereof, or one or more antigen binding domains to MOSPD2, specifically binds to MOSPD2 with a Kₒₙ of from about 10³ l/Ms to about 10⁶ l/Ms, or any range of values thereof (e.g., from about 10³ l/Ms to about 10⁵ l/Ms, from about 10⁴ l/Ms to about 10⁵ l/Ms, from about 10⁴ l/Ms to about 10⁶ l/Ms, from about 10⁵ l/Ms to about 10⁶ l/Ms, or from about 10³ l/Ms to about 10⁴ l/Ms). In other embodiments, the bispecific antibody or antigen binding fragment thereof, or one or more antigen binding domains to MOSPD2, has a Kₒₙ of about 10³ l/Ms, about 10⁴ l/Ms, about 10⁵ l/Ms, or about 10⁶ l/Ms.

In some embodiments, the bispecific antibody or antigen binding fragment thereof, or one or more antigen binding domains to MOSPD2, specifically binds to MOSPD2 with a K_{off} of from about 10⁻³ l/s to about 10⁻⁶ l/s, or any range of values thereof (e.g., from about 10⁻³ l/s to about 10⁻⁵ l/s, from about 10⁻⁴ l/s to about 10⁻⁵ l/s, from about 10⁻⁴ l/s to about 10⁻⁶ l/s, from about 10⁻⁵ l/s to about 10⁻⁶ l/s, or from about 10⁻³ l/s to about 10⁻⁴ l/s). In other embodiments, the bispecific antibody or antigen binding fragment thereof, or one or more antigen binding domains to MOSPD2, has a K_{off} of about 10⁻³ l/s, about 10⁻⁴ l/s, about 10⁻⁵ l/s, or about 10⁻⁶ l/s.

In some embodiments, the bispecific antibody or antigen binding fragment thereof, or one or more antigen binding domains to MOSPD2, specifically binds to one or more of the following amino acid regions of MOSPD2, numbered according to SEQ ID NO:1: about 505 to about 515, about 500 to about 515, about 230 to about 240, about 510 to about 520, about 210 to about 220, about 15 to about 25, about 505 to about 520, about 505 to about 515, about 90 to about 100, about 505 to about 525, about 230 to about 245, about 505 to about 510, about 130 to about 140, about 220 to about 230, about 15 to about 30, about 80 to about 95, about 40 to about 50, about 460 to about 475, about 340 to about 350, about 500 to about 515, about 460 to about 470, about 325 to about 335, about 20 to about 35, about 215 to about 225, about 510 to about 520, about 175 to about 190, about 500 to about 510, about 505 to about 530, about 60 to about 75, about 500 to about 520, about 145 to about 160, about 502 to about 515, about 85 to about 100, about 205 to about 220, about 175 to about 190, about 500 to about 505, about 500 to about 525, about 495 to about 505, about 495 to about 510, about 190 to about 200, about 190 to about 198, about 502 to about 515, about 1 to about 60, about 80 to about 240, about 90 to about 235, about 330 to about 445, about 330 to about 430, about 495 to about 515, about 145 to about 240, about 145 to about 220, about 145 to about 200, about 160 to about 240, about 160 to about 220, about 160 to about 200, about 175 to about 240, about 175 to about 220, about 175 to about 200, about 170 to about 190, about 178 to about 185, about 85 to about 140, about 85 to about 130, about 90 to about 140, about 90 to about 130, about 95 to about 140, about 95 to about 130, about 100 to about 130, about 100 to about 140, about 110 to about 130, or about 115 to about 127.

In some embodiments, the bispecific antibody or antigen binding fragment thereof, or one or more antigen binding domains to MOSPD2, specifically binds to one or more of the following amino acid regions of MOSPD2, numbered according to SEQ ID NO:1: about 508 to about 517, about 501 to about 514, about 233 to about 241, about 509 to about 517, about 212 to about 221, about 13 to about 24, about 505 to about 517, about 505 to about 514, about 89 to about 100, about 506 to about 517, about 233 to about 245, about 504 to about 514, about 128 to about 136, about 218 to about 226, about 15 to about 24, about 83 to about 96, about 42 to about 50, about 462 to about 474, about 340 to about 351, about 504 to about 517, about 462 to about 470, about 327 to about 337, about 21 to about 32, about 217 to about 226, about 510 to about 517, about 178 to about 190, about 497 to about 509, about 504 to about 516, about 64 to about 77, about 504 to about 515, about 147 to about 159, about 503 to about 515, about 88 to about 97, about 208 to about 218, about 178 to about 191, about 502 to about 515, about 503 to about 516, about 497 to about 505, about 500 to about 509, about 189 to about 202, about 189 to about 197, about 505 to about 516, about 1 to about 63, about 82 to about 239, about 93 to about 234, about 327 to about 445, about 327 to about 431, and about 497 to about 517.

Antibodies that specifically bind to MOSPD2 that can be used in the bispecific antibodies of the invention have been described in the literature, for example, in Int'l Pub. No. WO 2017/021855 and Int'l Pub. No. WO 2017/021857, which are incorporated by reference herein in their entireties.

Antibodies that specifically bind to a T cell- or NK cell-specific receptor molecule (e.g., CD3) that can be used in the bispecific antibodies of the invention have been described in the literature, for example, U.S. Pat. Nos. 7,112,324; 7,262,276; 7,635,472; 9,249,217; and 9,676,858, which are incorporated by reference herein in their entireties.

In addition, methods for obtaining a bispecific antibody or antigen binding fragment thereof of the invention, using antibodies and antigen binding domains that specifically bind to MOSPD2 and antibodies and antigen binding domains that specifically bind to a T cell- or NK cell-specific receptor molecule, have also been described in the literature, for example, in U.S. Pat. Nos. 7,112,324; 7,262,276; 7,635,472; 9,249,217; and 9,676,858, which are incorporated by reference herein in their entireties.

In some embodiments, the present invention relates to a nucleic acid encoding a bispecific antibody or antigen binding fragment thereof described herein.

In some embodiments, the present invention relates to an expression vector comprising a nucleic acid encoding a bispecific antibody or antigen binding fragment thereof described herein.

### Pharmaceutical Compositions and Kits

Other embodiments of the invention relate to a pharmaceutical composition comprising a bispecific antibody or antigen binding fragment thereof that specifically binds to MOSPD2 and to a T cell- or NK cell-specific receptor molecule.

In some embodiments, the pharmaceutical composition comprises a bispecific antibody or antigen binding fragment thereof described herein, and a pharmaceutically acceptable carrier. The term “pharmaceutically acceptable carrier” includes, but is not limited to, excipients, lubricants, buffering agents, antibacterial agents, bulking agents (e.g., mannitol), antioxidants (e.g., ascorbic acid or sodium bisulfite), diluents, adjuvants, vehicles, and the like.

In some embodiments, the pharmaceutical composition is suitable for systemic, local, nasal, oral, intra-peritoneal, intra-tumor, parenteral, transmucosal, rectal, buccal, inhalation, intravenous, intramuscular, or subcutaneous administration.

In some embodiments, the pharmaceutical composition contains a therapeutically effective amount of a bispecific antibody or antigen binding fragment thereof that specifically binds to MOSPD2 and to a T cell- or NK cell-specific receptor molecule, for example, from about 1 μg/ml to about 10 μg/ml, or any range of values thereof (e.g., from about 2 μg/ml to about 10 μg/ml, from about 3 μg/ml to about 10 μg/ml, from about 4 μg/ml to about 10 μg/ml, from about 5 μg/ml to about 10 μg/ml, from about 6 μg/ml to about 10 μg/ml, from about 7 μg/ml to about 10 μg/ml, from about 8 μg/ml to about 10 μg/ml, from about 9 μg/ml to about 10 μg/ml, from about 1 μg/ml to about 9 μg/ml, from about 2 μg/ml to about 9 μg/ml, from about 3 μg/ml to about 9 μg/ml, from about 4 μg/ml to about 9 μg/ml, from about 5 μg/ml to about 9 μg/ml, from about 6 μg/ml to about 9 μg/ml, from about 7 μg/ml to about 9 μg/ml, from about 8 μg/ml to about 9 μg/ml, from about 1 μg/ml to about 8 μg/ml, from about 2 μg/ml to about 8 μg/ml, from about 3 μg/ml to about 8 μg/ml, from about 4 μg/ml to about 8 μg/ml, from about 5 μg/ml to about 8 μg/ml, from about 6 μg/ml to about 8 μg/ml, from about 7 μg/ml to about 8 μg/ml, from about 1 μg/ml to about 7 μg/ml, from about 2 μg/ml to about 7 μg/ml, from about 3 μg/ml to about 7 μg/ml, from about 4 μg/ml to about 7 μg/ml, from about 5 μg/ml to about 7 μg/ml, from about 6 μg/ml to about 7 μg/ml, from about 1 μg/ml to about 6 μg/ml, from about 2 μg/ml to about 6 μg/ml, from about 3 μg/ml to about 6 μg/ml, from about 4 μg/ml to about 6 μg/ml, from about 5 μg/ml to about 6 μg/ml, from about 1 μg/ml to about 5 μg/ml, from about 2 μg/ml to about 5 μg/ml, from about 3 μg/ml to about 5 μg/ml, from about 4 μg/ml to about 5 μg/ml, from about 1 μg/ml to about 4 μg/ml, from about 2 μg/ml to about 4 μg/ml, from about 3 μg/ml to about 4 μg/ml, from about 1 μg/ml to about 3 μg/ml, from about 2 μg/ml to about 3 μg/ml, or from about 1 μg/ml to about 2 μg/ml).

In some embodiments, the therapeutically effective amount is about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, or about 10 μg/ml.

In some embodiments, the therapeutically effective amount is from about 10 mg/kg to about 40 mg/kg, or any range of values thereof (e.g., from about 15 mg/kg to about 40 mg/kg, from about 20 mg/kg to about 40 mg/kg, from about 25 mg/kg to about 40 mg/kg, from about 30 mg/kg to about 40 mg/kg, from about 35 mg/kg to about 40 mg/kg, from about 10 mg/kg to about 35 mg/kg, from about 15 mg/kg to about 35 mg/kg, from about 20 mg/kg to about 35 mg/kg, from about 25 mg/kg to about 35 mg/kg, from about 30 mg/kg to about 35 mg/kg, from about 10 mg/kg to about 30 mg/kg, from about 15 mg/kg to about 30 mg/kg, from about 20 mg/kg to about 30 mg/kg, from about 25 mg/kg to about 30 mg/kg, from about 10 mg/kg to about 25 mg/kg, from about 15 mg/kg to about 25 mg/kg, from about 20 mg/kg to about 25 mg/kg, from about 10 mg/kg to about 20 mg/kg, from about 15 mg/kg to about 20 mg/kg, or from about 10 mg/kg to about 15 mg/kg).

In some embodiments, the therapeutically effective amount is about 10 mg/kg, about 15 mg/kg, about 20 mg/kg, about 25 mg/kg, about 30 mg/kg, about 35 mg/kg, or about 40 mg/kg.

In some embodiments, the present invention relates to a kit comprising (i) a bispecific antibody or antigen binding fragment described herein or a pharmaceutical composition of described herein, and (ii) instructions for use. In some embodiments, the use in the instructions is one or more of the methods of use described herein.

### Methods of Use and Production

In some embodiments, the present invention relates to a method of treating or preventing cancer in a subject comprising administering to the subject a bispecific antibody or antigen binding fragment thereof described herein or a pharmaceutical composition described herein in an effective amount to treat or prevent cancer. Effective amounts of a bispecific antibody or antigens binding fragment thereof are described elsewhere herein.

In some embodiments, the present invention relates to a method of treating or preventing cancer metastasis in a subject comprising administering to the subject a bispecific antibody or antigen binding fragment thereof described herein or a pharmaceutical composition described herein in an effective amount to treat or prevent cancer metastasis.

In some embodiments, the cancer of any of the methods described herein is bladder cancer, brain cancer, breast cancer, colon cancer, rectal cancer, kidney cancer, liver cancer, lung cancer, esophageal cancer, gall-bladder cancer, ovarian cancer, pancreatic cancer, stomach cancer, cervical cancer, thyroid cancer, prostate cancer, skin cancer, hematopoietic cancer, tongue cancer, cancer of mesenchymal origin, cancer of central or peripheral nervous system, endometrial cancer, head and neck cancer, glioblastoma, or malignant ascites.

In some embodiments, the lung cancer is small-cell lung cancer or non-small-cell lung cancer.

In some embodiments, the skin cancer is squamous cell carcinoma, basal cell cancer, melanoma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, or angiosarcoma.

In some embodiments, the hematopoietic cancer is a hematopoietic cancer of lymphoid lineage. In some embodiments, the hematopoietic cancer of lymphoid lineage is leukemia, acute lymphocytic leukemia, chronic lymphocytic leukemia, acute lymphoblastic leukemia, B cell lymphoma, T cell lymphoma, Hodgkin's lymphoma, non-Hodgkin's lymphoma, hairy cell lymphoma, or Burkitt's lymphoma. In some embodiments, the hematopoietic cancer is a hematopoietic cancer of myeloid lineage. In some embodiments, the hematopoietic cancer of myeloid lineage is acute myelogenous leukemia, chronic myelogenous leukemia, myelodysplastic syndrome, or promyelocytic leukemia.

In some embodiments, the cancer of mesenchymal origin is fibrosarcoma, rhabdomyosarcoma, soft tissue sarcoma, or bone sarcoma.

In some embodiments, the cancer of the central or peripheral nervous system is astrocytoma, neuroblastoma, glioma, or schwannomas.

In some embodiments, the cancer is anal cancer, bone cancer, gastrointestinal stomal cancer, gestational trophoblastic disease, Hodgkin's lymphoma, Kaposi sarcoma, keratoacanthoma, malignant mesothelioma, multicentric castleman disease, multiple myeloma and other plasma cell neoplasms, myeloproliferative neoplasms, neuroblastoma, non-Hodgkin's lymphoma, osteosarcoma, ovarian, fallopian tube, or primary peritoneal cancer, penile cancer, retinoblastoma, rhabdomyosarcoma, seminoma, soft tissue sarcoma, stomach (gastric) cancer, testicular cancer, teratocarcinoma, thyroid follicular cancer, vaginal cancer, vulvar cancer, Wilms tumor and other childhood kidney cancers, or xeroderma pigmentosum.

In some embodiments, the method of treating or preventing cancer or cancer metastasis further comprises administering to the subject an effective amount of an anticancer drug. In some embodiments, the anticancer drug is Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and I 131 Iodine Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPDX, Carboplatin, Carboplatin-Taxol, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, Chlorambucil-Prednisone, CHOP, Cisplatin, Clafen (Cyclophosphamide), Clofarabine, CMF,Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), Folfiri, Folfiri-Bevacizumab, Folfiri-Cetuximab, Folfirinox, Folflox, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, OEPA, Ofatumumab, OFF, Olaparib, Omacetaxine Mepesuccinate, Oncaspar (Pegaspargase), Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and I 131 Iodine Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate), or a combination thereof.

In some embodiments, the present invention relates to a method for inhibiting or reducing tumor cells in a subject comprising administering to the subject a bispecific antibody or antigen binding fragment thereof described herein or a pharmaceutical composition described herein. In some embodiments, the number of tumor cells are reduced by at least about 10%, at least about 20%, at least about 30%, at least about 40%, at least about 50%, at least about 60%, at least about 70%, at least about 80%, at least about 90%, or at least about 100%, compared to a control or reference value.

In some embodiments, the present invention relates to a method of increasing the production of cytokines by cells expressing CD3 in a subject comprising administering to the subject a bispecific antibody or antigen binding fragment thereof described herein or a pharmaceutical composition described herein.

In some embodiments, the present invention relates to a method of increasing IL-2, CD69, and/or IFN-γ production or concentration in a T cell comprising contacting the T cell with a bispecific antibody or antigen binding fragment thereof described herein or a pharmaceutical composition described herein. In some embodiments, IFN-γ production increases by at least about 100%, at least about 200%, at least about 300%, at least about 400%, at least about 500%, at least about 600%, at least about 700%, at least about 800%, at least about 900%, or at least about 1000% compared to a control or reference value. In some embodiments, the IFN-γ concentration increases by at least about 1000 pg/ml, at least about 2000 pg/ml, at least about 3000 pg/ml, at least about 4000 pg/ml, at least about 5000 pg/ml, at least about 6000 pg/ml, at least about 7000 pg/ml, at least about 8000 pg/ml, at least about 9000 pg/ml, or at least about 10000 pg/ml compared to a control or reference value. In some embodiments, CD69 production increases by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, or at least about 30% compared to a control or reference value.

In some embodiments, the present invention relates to a method of stimulating an immune response in a subject comprising administering to the subject a bispecific antibody or antigen binding fragment thereof described herein or a pharmaceutical composition described herein.

In some embodiments, the present invention relates to a method of stimulating a T cell-mediated cytotoxic immune response against a cancer cell in a subject comprising administering to the subject a bispecific antibody or antigen binding fragment thereof described herein or a pharmaceutical composition described herein.

In some embodiments, the present invention relates to a method of increasing T cell proliferation comprising contacting a T cell with a bispecific antibody or antigen binding fragment thereof described herein or a pharmaceutical composition described herein.

In some embodiments, the present invention relates to a method of reducing or depleting the number of T regulatory cells in a tumor of a subject comprising administering to the subject a bispecific antibody or antigen binding fragment thereof described herein or a pharmaceutical composition described herein.

In some embodiments, the present invention relates to a method for the prediction, diagnosis, or prognosis of cancer or cancer metastasis in a subject comprising determining the expression level of MOSPD2 in a sample of the subject using a bispecific antibody or antigen binding fragment thereof described herein. In some embodiments, the method comprises (i) determining or quantifying the expression level of MOSPD2 in a sample of the subject using a bispecific antibody or antigen binding fragment thereof described herein, and (ii) comparing the expression level obtained in step (i) with a control or reference value, wherein an increased expression level of MOSPD2 with respect to the control or reference value is indicative of cancer, an increased risk of developing cancer, or a poor cancer prognosis.

In some embodiments, the present invention relates to a method for the prediction, diagnosis, or prognosis of tumor progression or tumor invasiveness in a subject comprising determining the expression level of MOSPD2 in a sample of the subject using a bispecific antibody or antigen binding fragment thereof of described herein. In some embodiments, the method comprises (i) determining or quantifying the expression level of MOSPD2 in a sample of the subject using a bispecific antibody or antigen binding fragment thereof described herein, and (ii) comparing the expression level obtained in step (i) with a control or reference value, wherein an increased expression level of MOSPD2 with respect to the control or reference value is indicative or a poor tumor progression or tumor invasiveness prognosis.

In some embodiments, the method for prediction, diagnosis, or prognosis further comprises one or more of the following steps:

instructing a laboratory to quantify the expression level of MOSPD2 in the sample;

obtaining a report of the expression level of MOSPD2 in the sample from a laboratory; and/or

administering a therapeutically effective amount of an inhibitor of MOSPD2 to the subject.

In some embodiments of the method for prediction, diagnosis, or prognosis, the sample is a tissue biopsy, tumor biopsy, or blood sample from the subject.

In some embodiments of the method for prediction, diagnosis, or prognosis, the control or reference value is the expression level of MOSPD2 in normal tissue or normal adjacent tissue (NAT).

In some embodiments of the method for prediction, diagnosis, or prognosis, the control or reference value is no detectable MOSPD2 expression or no significant MOSPD2 expression.

In some embodiments, the present invention relates to a method for treating or preventing a MOSPD2-expressing tumor in a subject comprising administering to the subject a bispecific antibody or antigen binding fragment thereof described herein or a pharmaceutical composition described herein in an effective amount to treat or prevent a MOSPD2 expressing tumor.

In some embodiments, the present invention relates to a method for treating or preventing a tumor having MOSPD2-expressing tumor associated macrophages in a subject comprising administering to the subject a bispecific antibody or antigen binding fragment thereof described herein or a pharmaceutical composition described herein in an effective amount to treat or prevent a tumor having MOSPD2-expressing tumor associated macrophages.

In some embodiments, the subject of any of the methods described herein is a mammal. In some embodiments, the subject of any of the methods described herein is a human. In some embodiments, the subject of any of the methods described herein is a veterinary animal (e.g., a dog, cat, horse, sheep, cow or goat).

In some embodiments, the present invention relates to a method for producing the bispecific antibody or antigen binding fragment thereof described herein comprising culturing a host cell transformed with a nucleic acid or expression vector described herein, and collecting and purifying the expressed bispecific antibody or antigen binding fragment thereof.

### EXAMPLES

Reference is now made to the following examples, which together with the above descriptions illustrate some embodiments of the invention in a non-limiting fashion.

### Materials and Methods

### MOSPD2 Silencing.

The human breast cancer cell line MDA-MB-231 (hereafter MDA-231) (HTB-26) and the human malignant melanoma cell line A2058 (CRL-11147) were purchased from the American Type Culture Collection (ATCC). The cells (2×10⁶ in 2 ml) were placed in a 15 ml tube. Lenti-virus particles expressing control short hairpin RNA (sh-RNA) (2×10⁵ viral particles) or human MOSPD2 sh-RNA (2×10⁶ viral particles) were applied to the cells, which were then spun for 60 min, 2000 rpm at room temperature in the presence of 8 μg/ml polybrene (Sigma, Israel). The cells were then seeded in a 6 well plate. After 72 hour, fresh medium containing puromycin (4 μg/ml Sigma, Israel) was added for the selection of transduced cells. For CRISPR-CAS9 mediated silencing, MDA-231 cells were transduced with CRISPR-CAS9 non-target control or CRISPR-CAS9 human MOSPD2 lenti-viral particles as described above. Single cell cloning was performed on transduced cells to isolate cells with silenced MOSPD2 protein expression and impaired migration.

### Western Blot

sh-control or sh-MOSPD2 Lenti-virus transduced A2058 or MDA-231 cells, or control or MOSPD2 CRISPR-CAS9 lenti-viral particles transduced MDA-231 cells (10⁶), were washed and resuspended in lysis buffer containing 1:100 dithiothreitol (DTT), phosphatase and protease inhibitors (Thermo Scientific). Samples were loaded onto a precast Criterion TGX gel (Bio-Rad, Hemel Hempstead, UK) and transferred onto a nitrocellulose membrane. Blots were blocked with 5% milk or bovine serum albumin (BSA) in Tris buffered saline and Tween 20 (TBST) for 1 hour, followed by incubation with primary and secondary antibodies. Membranes were developed using an ECL kit (Thermo Scientific). The following antibodies were used for immunoblotting:

Primary antibodies: Rabbit anti-MOSPD2 (1:5000) generated by Vascular Biogenics Ltd. Phospho extracellular-regulated kinase (p-ERK1/2) (Thr 183 and Tyr 185, 1:4000) was purchased from Sigma (Israel). Phospho-AKT (Ser 473, 1:1000) was purchased from Cell Signaling. Phospho-FAK (1:2000) was purchased from Abcam (Cambridge, UK). Heat shock protein (HSP) 90 (1:1000) was purchased from Santa Cruz Biotechnology (Dallas, Tex.).

Secondary antibodies: Horseradish peroxidase (HRP) donkey anti-rabbit (1:5000) and HRP goat anti-mouse (1:5000) antibodies were purchased from Jackson ImmunoResearch (West Grove, Pa.).

### Q-PCR

To determine silencing efficacy, RNA was extracted from sh-control and sh-MOSPD2 Lenti-virus transduced MDA-231 cells using RNeasy mini kit (Qiagen, ValenVBa, Calif.). For cDNA preparation, 2 μg of RNA was combined with qScript reaction mix and qScript reverse transcriptase (Quanta Bioscience, Gaithersburg, Md.). The reaction was placed in a thermal cycler (BioRad, Hercules, Calif.) and a run program was set according to the manufacturer instructions. Real-time PCR reactions were performed on an Applied Biosystems 7300 real time PCR system (Grand Island, N.Y.) using sets of primers for human MOSPD2, 28S to normalize RNA levels (BIOSEARCH TECHNOLOGIES, Petaluma, Calif.) and SYBR Green PCR Master Mix (Applied Biosystems, Warrington, UK).

### Immunohistochemistry Staining

To assess the expression level of MOSPD2 in cancer tissues, Biomax arrays (US Biomax Rockville, Md.) for breast cancer (T088B and BR2028a), for liver cancer (BC03116a), and for multiple organ tumor (MC6163) were stained with the rabbit anti-MOSPD2 antibody or control rabbit IgG (R&D Systems Cat # AB-105-C) followed by incubation with anti-Rabbit HRP (Cat #0399 DAKO, Denmark).

### Example 1

### MOSPD2 and Migration of Metastatic Cell Lines

In order to assess the role of MOSPD2 in cancer cell migration, MOSPD2 expression in two metastatic cell lines, A2058 melanoma and MDA-231 breast cancer, was silenced using sh-control or sh-MOSPD2 lenti-virus particles as described in Int'l Pub. No. 2017/021857.

In particular, sh-control or sh-MOSPD2 transduced A2058 or MDA-231 cells (3×10⁵) previously starved for 3 hours in 0.5% FBS/RPMI-1640 were seeded in the upper chamber of a QCM 24-well, 5 μm pore, migration assay plate (Corning-Costar, Corning, N.Y.), followed by incubation for 24 hours in the presence of 10% FBS/RPMI-1640 and EGF (200 ng/ml, Peprotech Israel) in the lower chamber. Subsequently, the cells which migrated to the lower compartment were stained with crystal violet before images were taken.

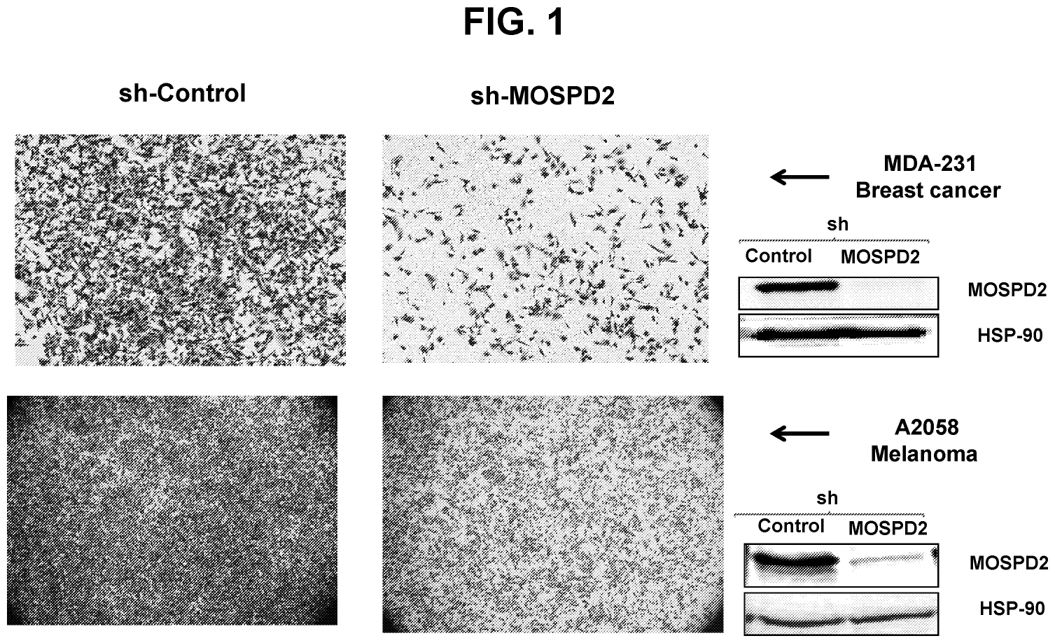

FIG. 1 demonstrates that sh-MOSPD2 lenti-virus particles have profoundly decreased protein expression and inhibited cancer cell migration in vitro.

### Example 2

### MOSPD2 and Cell Proliferation

To determine whether the inhibitory effect on cell migration subsequent to MOSPD2 silencing is secondary to fundamental cell function such as proliferation, sh-control or sh-MOSPD2 lenti-virus particle transduced MDA-231 breast cancer cells were tested for proliferation over a period of 3 days as described in Intl Pub. No. 2017/021857.

Specifically, sh-control or sh-MOSPD2 lenti-virus transduced MDA-231 cells were seeded in 6 well plates (10⁴ per well). The cells were counted by FACS every 24 hours in triplicates for 3 consecutive days.

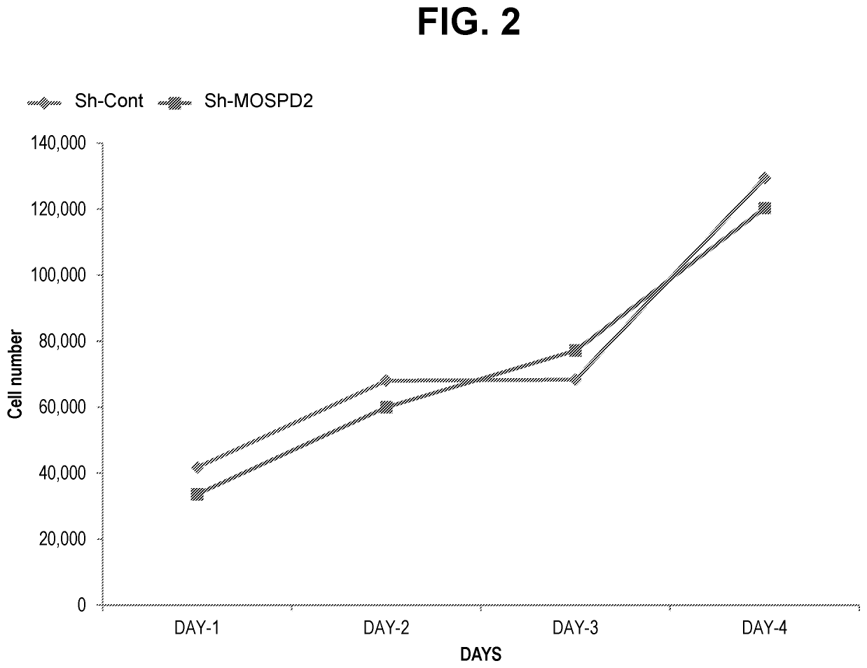

The data shown in FIG. 2 indicate that MOSPD2 is not essential for the proliferation of these cells, suggesting a regulatory role for MOSPD2 specifically in migration.

### Example 3

### MOSPD2 and Cell Metastasis

To assess the role of MOSPD2 in disseminating cancer cells to organs beyond the original site of cancer, the extent of lung metastasis in sh-control or sh-MOSPD2 lenti-virus particle-transduced MDA-231 breast cancer cells were adoptively transferred into immune-deficient mice as described in Intl Pub. No. 2017/021857. In another model in which the site of inception occurs in the breast, immunodeficient mice were inoculated with sh-control or sh-MOSPD2 lenti-virus particle-transduced MDA-231 breast cancer cells in the mammary fat pad.

Pathological examination: Histology slides were stained with hematoxylin/eosin (H&E). Formalin-fixed tissue was dehydrated, embedded in paraffin, and sectioned at 4 μm thickness. The H&E staining was calibrated on a Leica staining module. The slides were warmed to 90° C. for 7 minutes and then processed according to a fully automated protocol. After sections were dewaxed and rehydrated, slides were stained for 7 minutes in Gill's Hematoxylin No. 3 (Surgipath), washed, dipped in acidic alcohol, and washed. After short dipping in 70% ethanol and 96% ethanol, slides were stained for 4 minutes in eosin (Sigma), and dehydrated in 96% ethanol and then twice in 100% ethanol for 1 minute each time. After a run on an automated stainer was completed, sections were cleared in xylene for 10 seconds and mounted with Entellan. Mean tumor area comprises the maximal lung tumor area measured for each mouse.

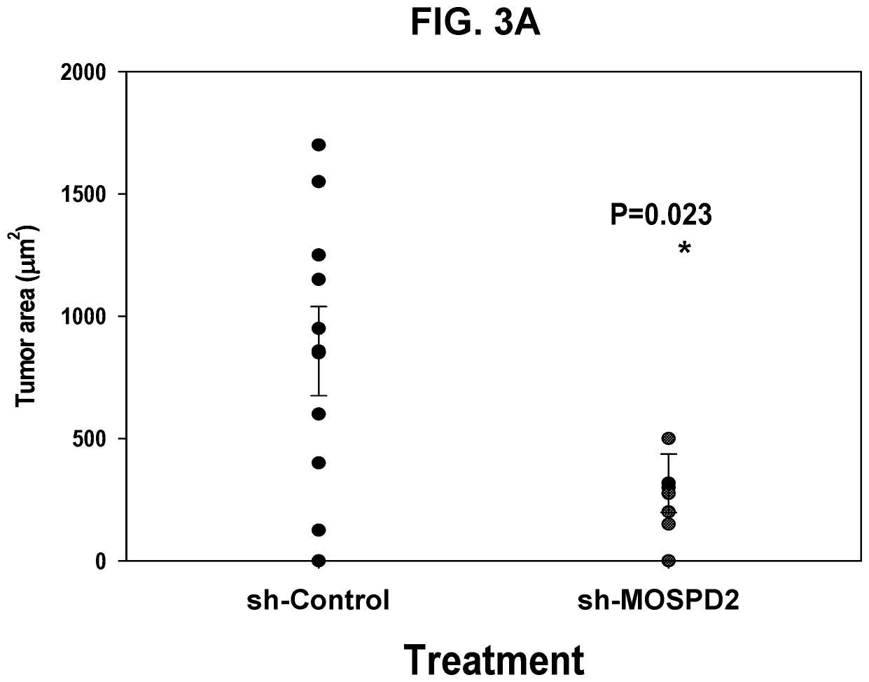

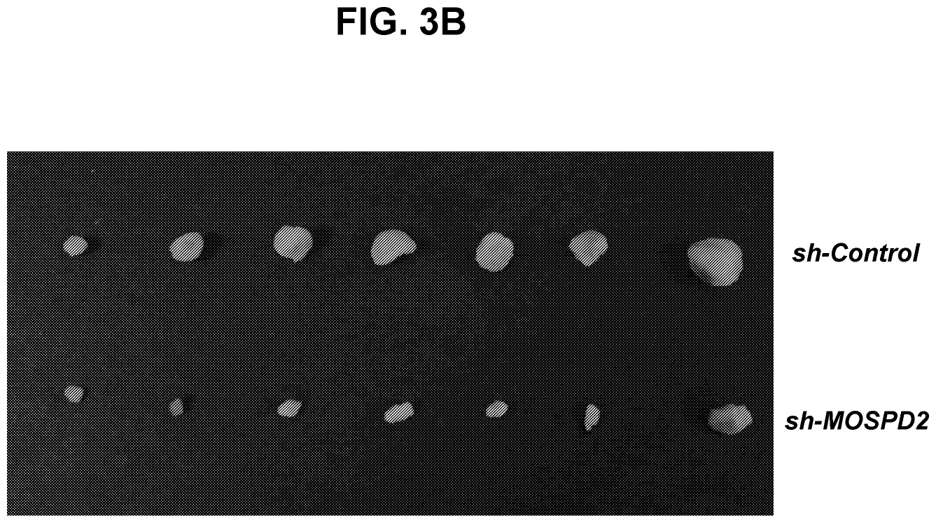

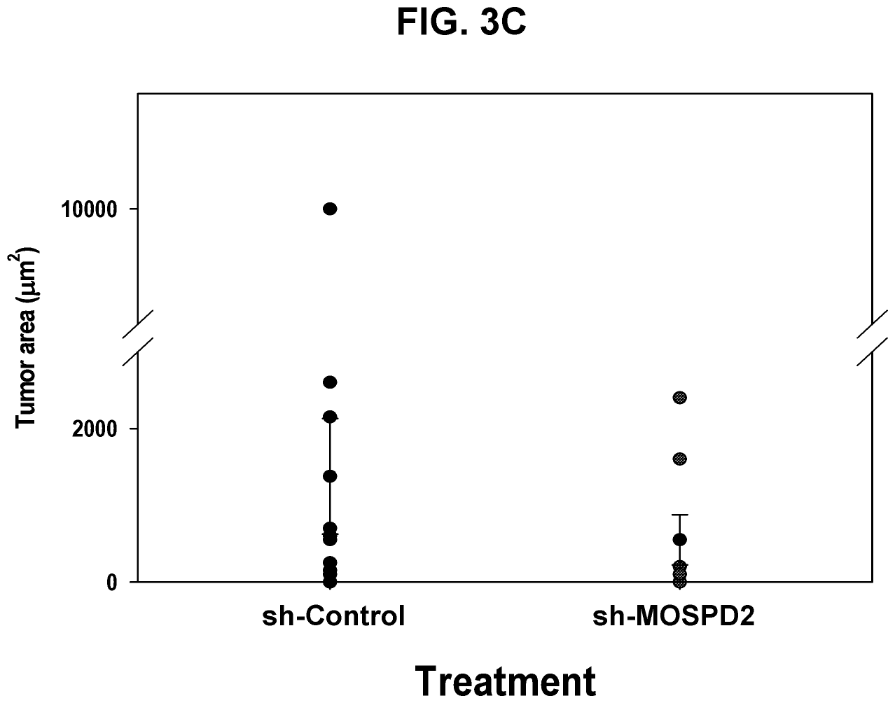

Systemic: 10⁶ sh-control or sh-MOSPD2 lenti-virus transduced MDA-231 cells were injected into the tail vein of 8 weeks old female SCID mice (C.B-17/IcrHsd-Prkdc^{scid}, Harlan Israel). Mice were sacrificed after 4 weeks. Lungs were excised for histopathologic examination. The results in FIG. 3A show that silencing MOSPD2 expression significantly (p=0.023) inhibits the presence of metastatic breast cancer cells in the lungs by more than 50% (metastasis area).

Orthotopic: 5×10⁶ sh-control or sh-MOSPD2 lenti-virus transduced MDA-231 cells were injected into the mammary fat pad of 8 weeks old female SCID mice (C.B-17/IcrHsd-Prkdc^{scid}, Harlan Israel). Mice were sacrificed after 10 weeks. Ipsilateral inguinal lymph node and the lungs were excised for examination. Macroscopic examination showed that the vast majority of lymph nodes excised from mice transferred with sh-control cells were overwhelmingly bigger than those from mice transferred with sh-MOSPD2 treated cells (FIG. 3B). Moreover, the mean metastasis area measured in the lungs of mice transferred with sh-MOSPD2 treated cells was reduced by more than 50% compared to the control group (FIG. 3C).

The ratio of MOSPD2 mRNA silencing in sh-MOSPD2 injected cells was ˜80%, as determined by Q-PCR as described in the Materials and Methods.

These results demonstrate that MOSPD2 plays a major role in breast cancer metastasis.

### Example 4

### MOSPD2 Expression in Various Types of Cancer

To determine whether MOSPD2 expression is associated with the transformation of cells from normal to cancerous, slides carrying normal and cancerous tissues were screened using anti-MOSPD2 antibody as described in the Materials and Methods section and in Intl Pub. No. 2017/021857.

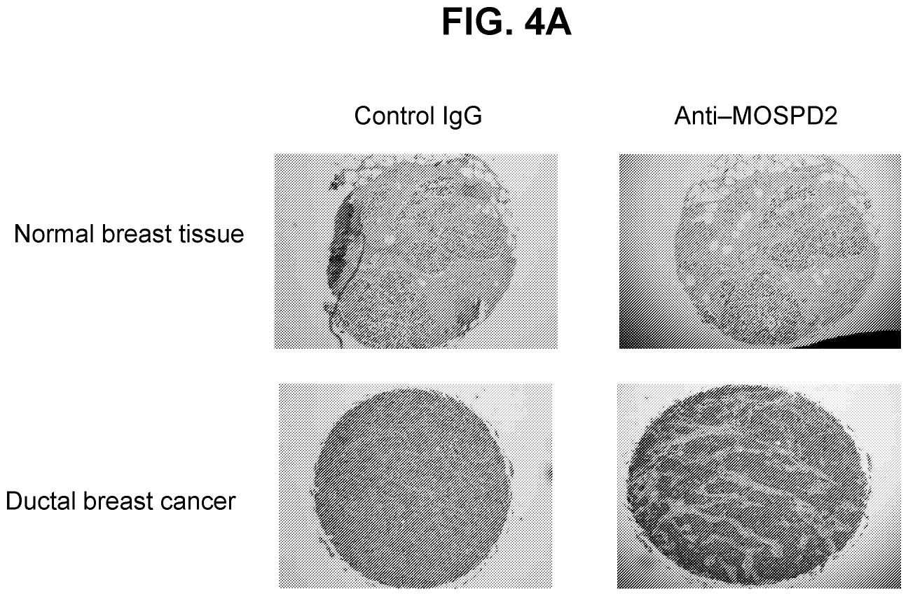

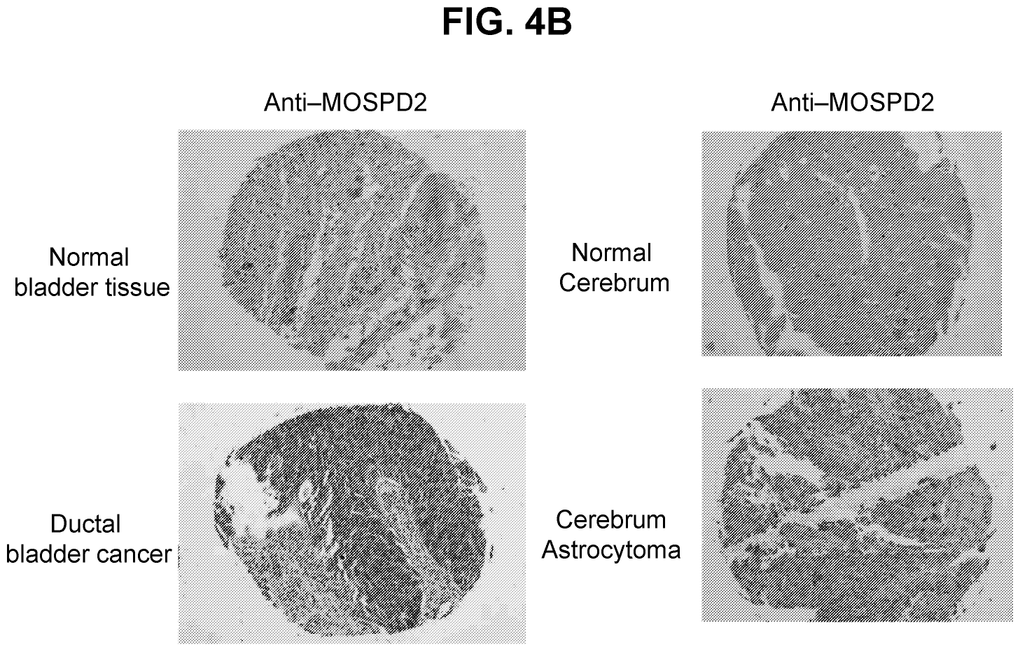

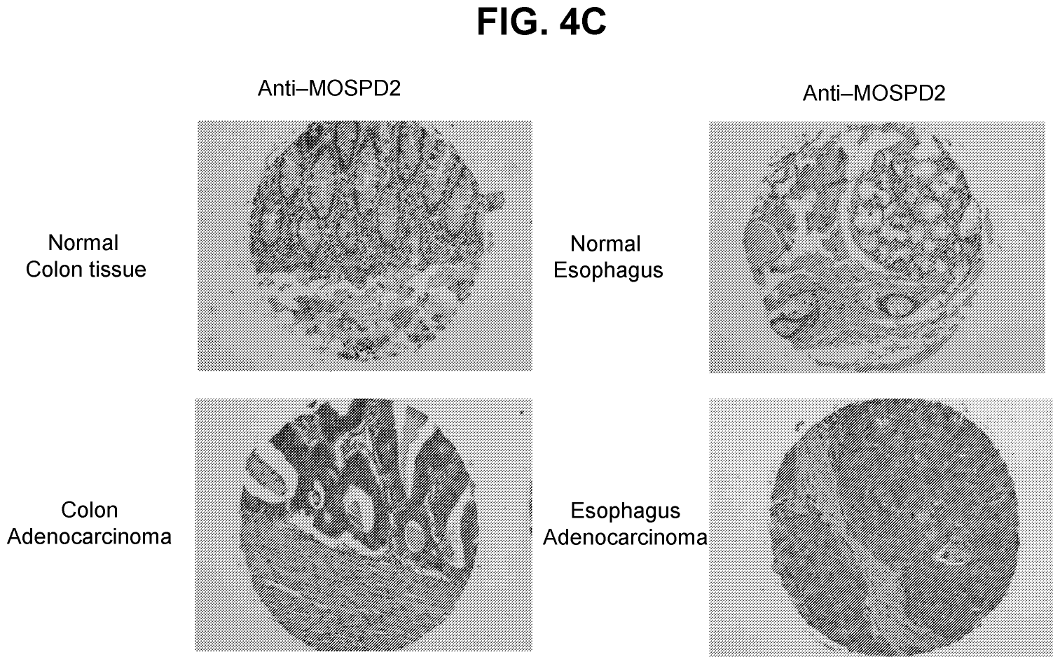

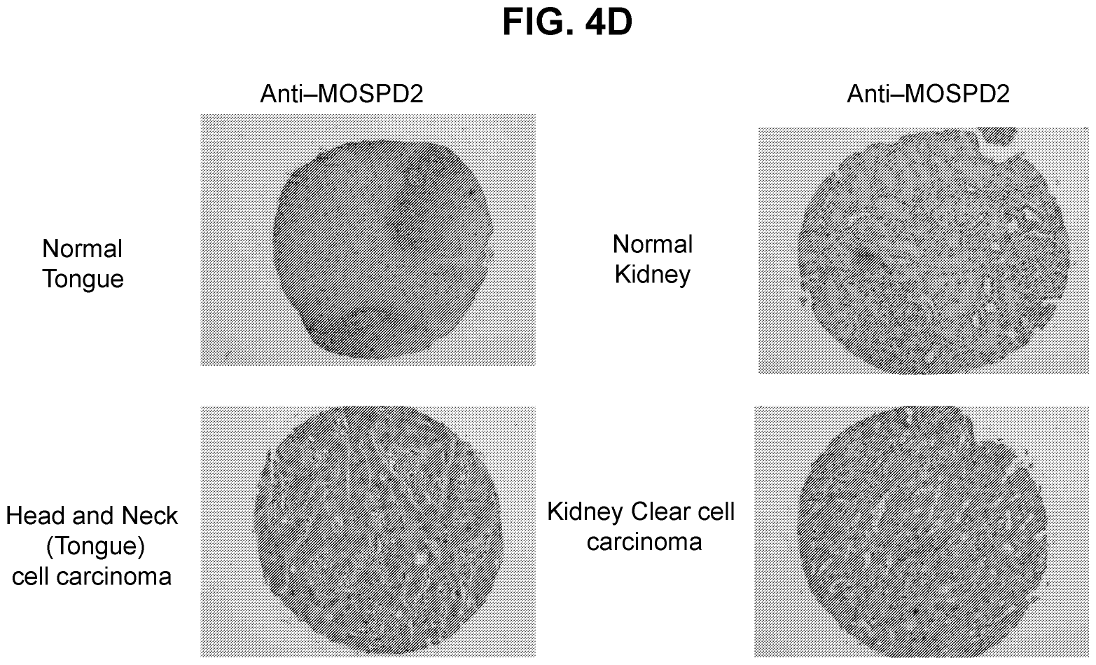

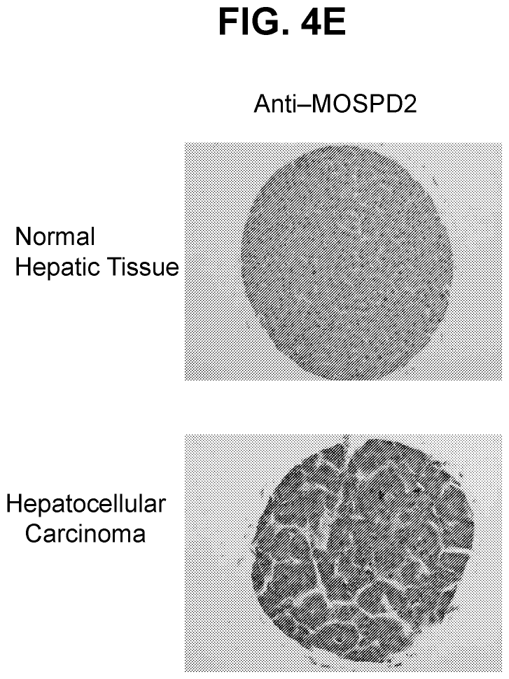

FIG. 4A shows representative staining of normal and cancerous breast tissue. While normal and cancerous breast tissues were negatively stained with control IgG antibody, anti-MOSPD2 antibody distinctively stained cancerous tissues only. Similarly, MOSPD2 is not expressed in normal bladder, brain, colon, esophagus, tongue, kidney and hepatic tissues, but is upregulated when these tissues turn cancerous (FIGS. 4B-4E). These results suggest that in various tissues, MOSPD2 expression is associated with transformation of normal tissue to cancerous tissue.

### Example 5

### MOSPD2 Gene Knockdown and Cancer Cell Migration

In vitro: To achieve sustainable knockdown of MOSPD2, MDA-231 breast cancer cells were transduced with lenti-viral particles that contain the CRISPR-CAS9 gene editing system as described in the Materials and Methods section and in Int'l Pub. No. 2017/021857. Control or MOSPD2 CRISPR-CAS9 lenti-viral particles transduced MDA-231 cells were tested for migration by methods described above. Control or MOSPD2 CRISPR-CAS9 lenti-viral particles transduced MDA-231 cells (3×10⁵) were seeded in the upper chamber, followed by incubation for 2-4 hours. Subsequently, the number of cells which migrated to the lower compartment was determined by FACS.

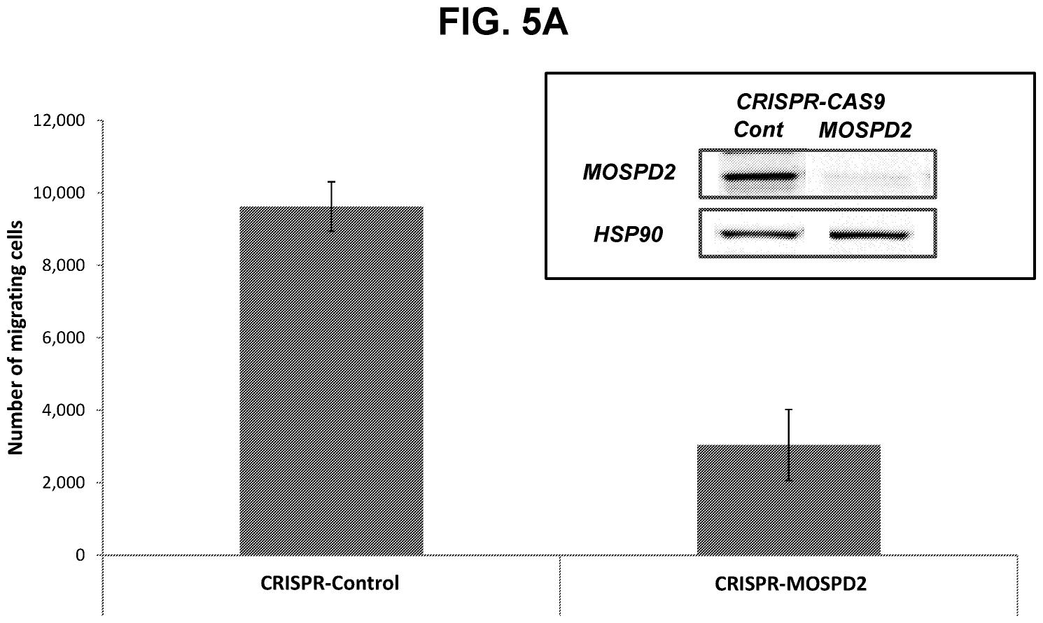

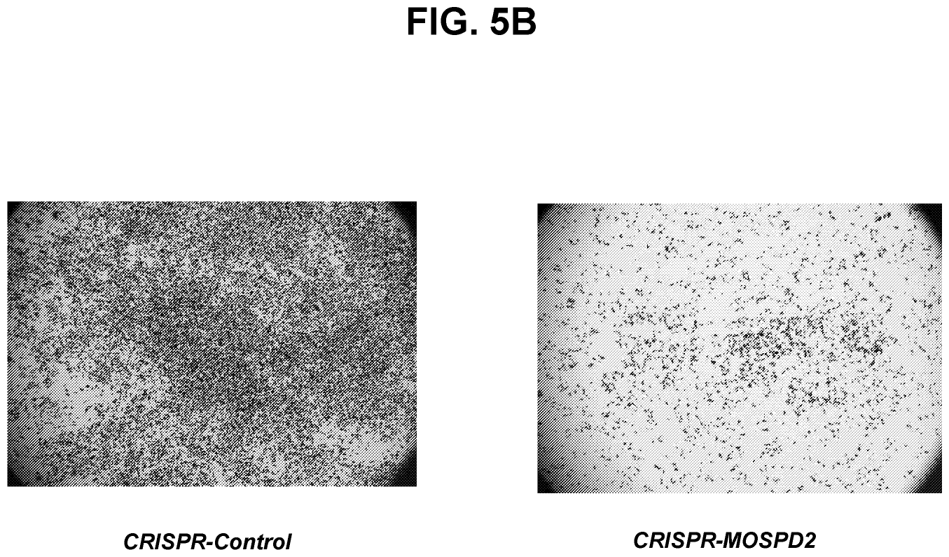

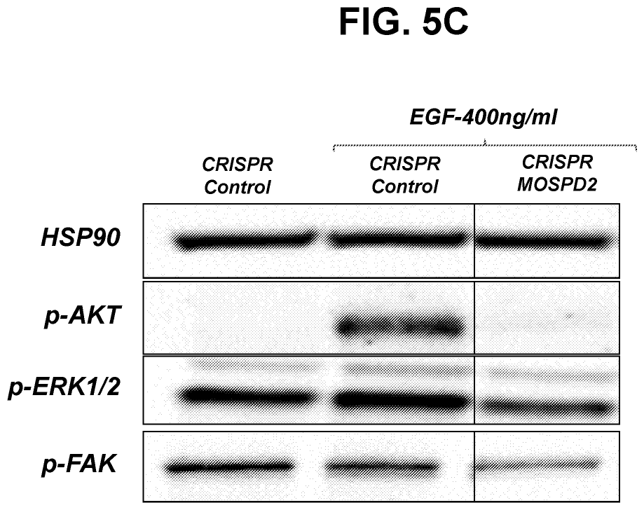

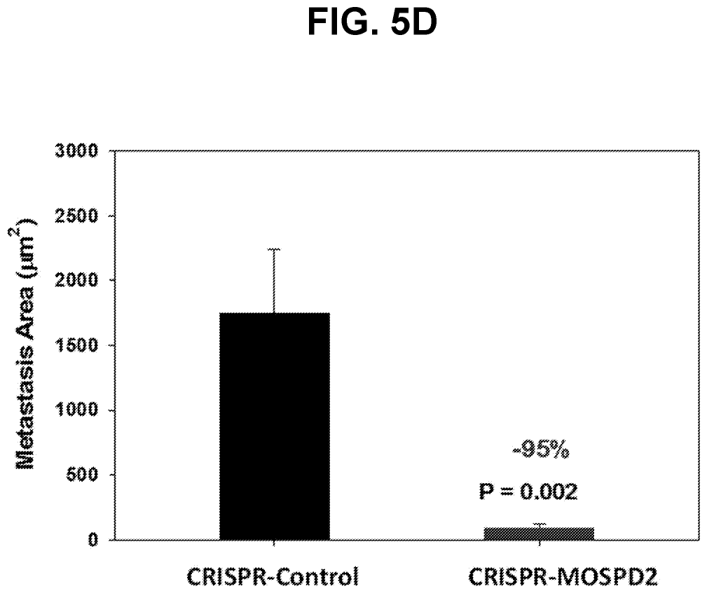

FIGS. 5A and 5B show that introducing the CRISPR-CAS9 system for MOSPD2 in MDA-231 cancer cells abolished protein expression and consequently profoundly inhibited migration of the cells in a trans-well assay.

To test the effects of MOSPD2 silencing by CRISPR-CAS9 on chemokine receptor-driven signaling events, phosphorylation levels of ERK, AKT and FAK were studied as described in the Materials and Methods. In accordance with the migration assay results, silencing MOSPD2 by the CRISPR-CAS9 system compared to control completely prevented phosphorylation of AKT and distinctly inhibited phosphorylation of ERK and FAK (see Western Blots in FIG. 5C) in cells exposed to EGF.

In vivo: 10⁶ CRISPR-control or CRISPR-MOSPD2 lenti-virus transduced MDA-231 cells were injected into the tail vein of 8 weeks old female SCID mice (C.B-17/IcrHsd-Prkdcscid, Harlan Israel). Mice were then sacrificed after 3 weeks. Lungs were excised for histopathologic examination. FIG. 5D shows that silencing MOSPD2 by the CRISPR-CAS9 system significantly inhibited the presence of metastatic breast cancer cells in the lungs by more than 95% (metastasis area).

### Example 6

### Anti-MOSPD2 Flab)2 mAb Specifically Binds to Endogenous MOSPD2 on Human Breast Cancer Cells

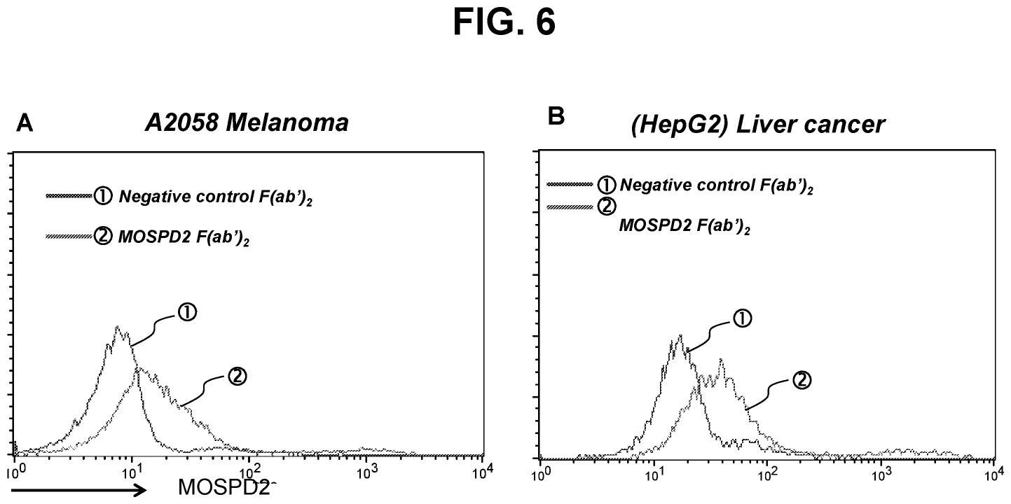

As described in Int'l Pub. No. 2017/021857, an anti-MOSPD2 F(ab′)₂ mAb was generated and tested for binding to surface expressed endogenous MOSPD2 on A2058 melanoma and HepG2 liver cancer cell lines. Cells were stained with anti-MOSPD2 F(ab′)₂ mAb and tested for binding to MOSPD2. FIGS. 6A-6B show that anti-MOSPD2 F(ab′)₂ mAb specifically binds to endogenous MOSPD2 on melanoma and liver cancer cells.

### Example 7

### Anti-MOSPD2 Flab)2 mAb Inhibits EGF-induced Migration of MDA-231 Cancer Cells

The effect of anti-MOSPD2 F(ab′)₂ mAb on EGF-induced migration of MDA-231 cancer cells was analyzed with trans-well migration as explained in Int'l Pub. No. 2017/021857. MDA-231 breast cancer cells (3×10⁵) were starved for 4-5 hr in RPMI medium containing 0.5% FCS and then incubated for 1 hr with anti-MOSPD2 F(ab′)₂ mAb. EGF was dissolved and placed in the lower chamber (400 ng/ml) of a QCM 24-well migration assay plate (8 μm pores) (Corning-Costar, Corning, N.Y.) which contained RPMI medium with 10% FCS. Cells were seeded in the upper chamber, followed by over-night incubation, after which the number of cells that migrated to the lower compartment was determined by FACS.

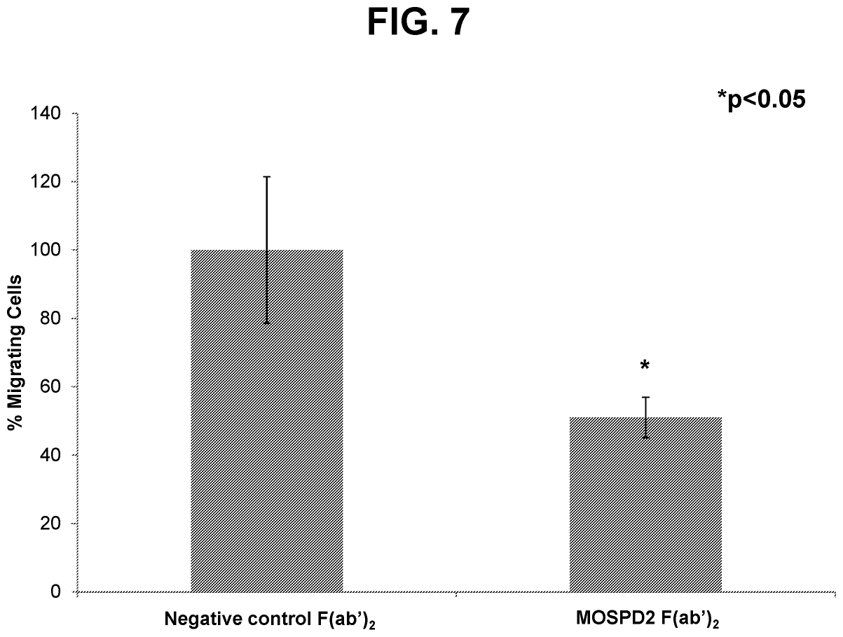

As shown in FIG. 7, anti-MOSPD2 F(ab′)₂ mAb significantly inhibited EGF-induced trans-well migration of MDA-231 breast cancer cells.

### Example 8

### Epitope Mapping of Anti-MOSPD2 Antibodies

To determine the epitope(s) that anti-MOSPD2 antibodies may specifically bind on human MOSPD2, binding affinities to various human MOSPD2 fragments are measured, as described herein, by capturing N-terminally biotinylated MOSPD2 fragments via a pre-immobilized streptavidin (SA) on a SA chip and measuring binding kinetics of anti-MOSPD2 antibodies titrated across the MOSPD2 surface (the BIAcore®3000™ surface plasmon resonance (SPR) system, Biacore, Inc., Piscataway N.J.). BIAcore assays are conducted in HBS-EP running buffer (10 mM HEPES pH 7.4, 150 mM NaCl, 3 mM EDTA, 0.005% v/v polysorbate P20). MOSPD2 surfaces are prepared by diluting the N-biotinylated MOSPD2 to a concentration of less than 0.001 mg/mL into HBS-EP buffer and injecting it across the SA sensor chip using variable contact times. Low capacity surfaces, corresponding to capture levels <50 response units (RU) are used for high-resolution kinetic studies, whereas high capacity surfaces (about 800 RU of captured MOSPD2) are used for concentration studies, screening, and solution affinity determinations.

Kinetic data is obtained by diluting antibody G1 Fab serially in two- or three-fold increments to concentrations spanning 1 μM-0.1 nM (aimed at 0.1-10 times estimated K_{D}). Samples are typically injected for 1 minute at 100 μL/min and dissociation times of at least 10 minutes are allowed. After each binding cycle, surfaces are regenerated with 25 mM NaOH in 25% v/v ethanol, which is tolerated over hundreds of cycles. An entire titration series (typically generated in duplicate) is fit globally to a 1:1 Langmuir binding model using the BIAevaluation program. This returns a unique pair of association and dissociation kinetic rate constants (respectively, Kₒₙ and K_{off}) for each binding interaction, whose ratio gives the equilibrium dissociation constant (K_{D}=K_{off}/Kₒₙ).

Anti-MOSPD2 antibodies may specifically bind to one or more of the following amino acid regions of human MOSPD2, numbered according to SEQ ID NO:1 (amino acid residues 1-518): 508-517, 501-514, 233-241, 509-517, 212-221, 13-24, 505-517, 505-514, 89-100, 506-517, 233-245, 504-514, 128-136, 218-226, 15-24, 83-96, 42-50, 462-474, 340-351, 504-517, 462-470, 327-337, 21-32, 217-226, 510-517, 178-190, 497-509, 504-516, 64-77, 504-515, 147-159, 503-315, 88-97, 208-218, 178-191, 502-515, 503-516, 497-505, 500-509, 189-202, 189-197, 505-516, 1-63, 82-239, 93-234, 327-445, 327-431, 497-517, 145-240, 145-220, 145-200, 160-240, 160-220, 160-200, 175-240, 175-220, 175-200, 170-190, 178-185, 85-140, 85-130, 90-140, 90-130, 95-140, 95-130, 100-130, 100-140, 110-130, or 115-127.

### Example 9

### Additional Anti-MOSPD2 Antibodies

Additional anti-MOSPD2 antibodies are generated that recognize one or more MOSPD2 epitopes, following the methodology described herein or in Int'l Pub. No. 2017/021857.

Briefly, portions of MOSPD2 identified herein or in Int'l Pub. No. 2017/021857 as MOSPD2 epitopes are fused to human Fc and immobilized on a solid support. A HuCAL® library (HuCAL PLATINUM® Platform; Bio-Rad AbD Serotec, GmnH) presented on phage particles is incubated with the immobilized antigen. Nonspecific antibodies are removed by extensive washing and specific antibody phages are eluted by adding a reducing agent. Antibody DNA is isolated as a pool and subcloned into an E. coli expression vector to generate bivalent F(ab′)₂ mAb. Colonies are picked and grown in a microtiter plate. The cultures are lysed to release the antibody molecules and screened for specific antigen binding by ELISA and FACS. Unique antibodies are expressed and purified using one-step affinity chromatography, and then tested again by ELISA and FACS for specificity.

### Example 10

### MOSPD2 Expression is Increased in Correlation with Tumor Grade in Various Types of Cancer

To determine whether MOSPD2 expression was associated with tumor progression, slides carrying normal and cancerous tissues in different tumor grades were screened using anti-MOSPD2 antibody as described in Int'l Pub. No. 2017/021857. MOSPD2 abundance was scored according to the staining intensity on a scale from 0 to 3. In cases where intra-heterogeneity staining within a single core was observed, the score of the area with the highest coverage was assigned.

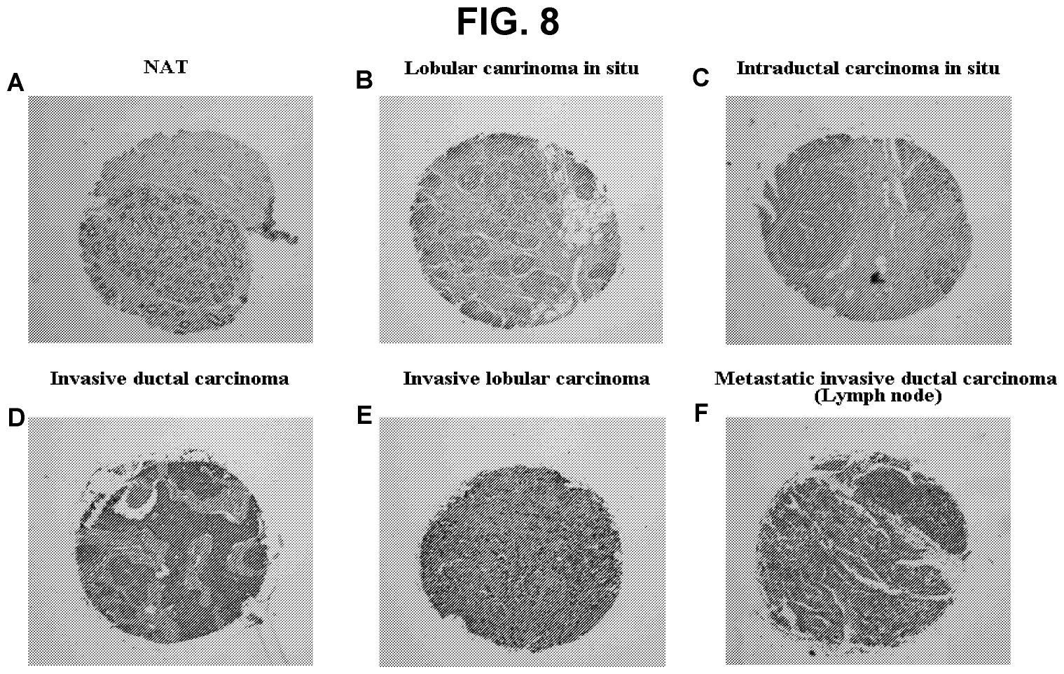

FIGS. 8A-8F show representative MOSPD2 staining in breast cancer and control tissue. Normal adjacent tissue (NAT) served as a negative control, and the escalating tumor stages included lobular carcinoma in situ (LCIS), intraductal carcinoma in situ (IDIS), invasive ductal carcinoma (IDC), invasive lobular carcinoma (ILC) and Metastatic invasive ductal carcinoma (MIDC). While representative NAT, LCIS and IDIS staining were negatively stained for MOSPD2, IDC, ILC and MIDC representative staining demonstrated intense positive MOSPD2 staining.

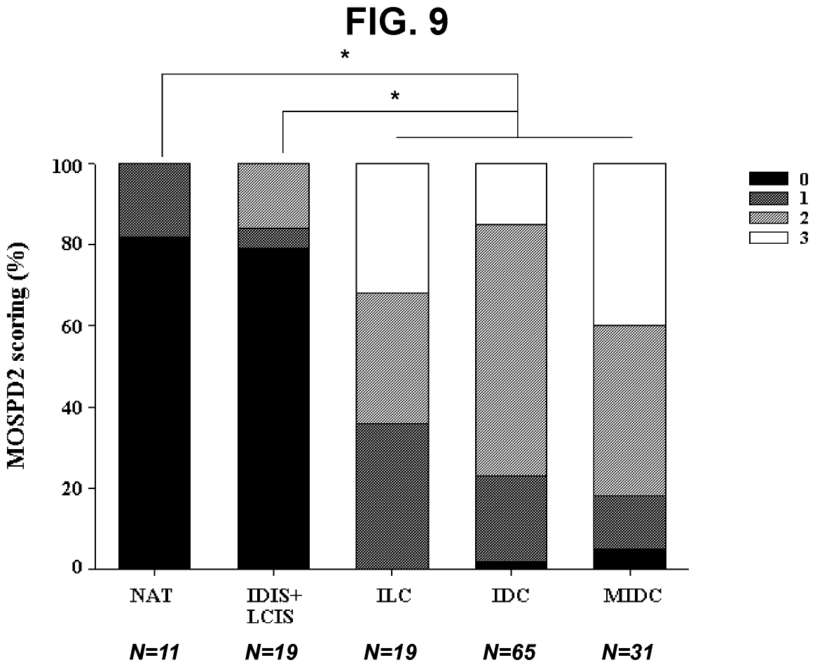

FIG. 9 demonstrates increased MOSPD2 staining intensity in invasive and metastatic breast cancer. Within NAT, only 18% percent (2/11) of samples showed a staining intensity of 1, while 21% (4/19) of in situ carcinoma samples (IDIS+LCIS) were scored 1 or 2. However, analysis of invasive and metastatic tissues demonstrated higher frequency in score of 2 and increased staining intensity up to score of 3, compared to NAT and in situ carcinoma (IDIS+LCIS). Thus, the percent of combined scores 2 and 3 for ILC, IDC and MIDC were 63% (12/19), 77% (50/65) and 81% (25/31), respectively.

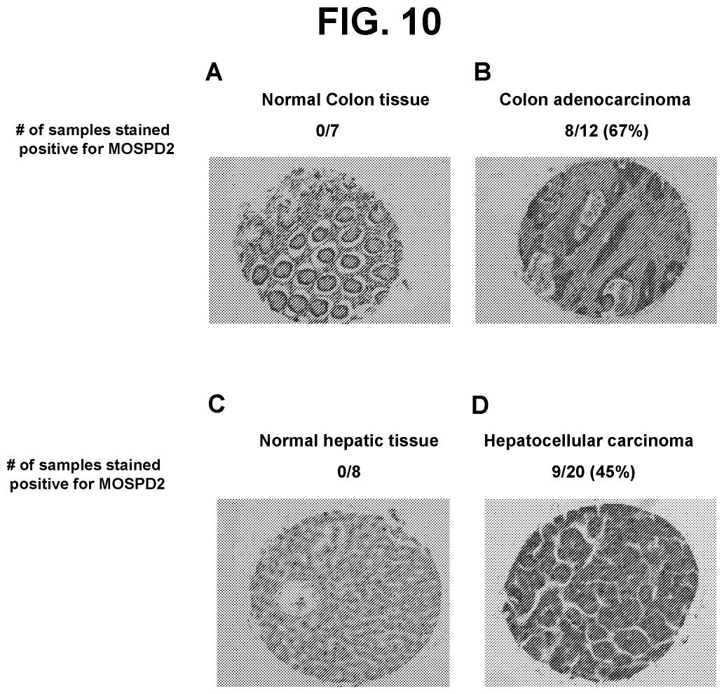

MOSPD2 expression correlated with the transformation of cells from normal to cancerous in colon and in hepatic tissues as well. FIGS. 10A-10D demonstrate that in 67% of colon cancer samples and in 45% of hepatocellular carcinoma samples tested, there was a positive MOSPD2 staining. No MOSPD2 staining (0%) was detected in the normal colon or liver tissues tested.

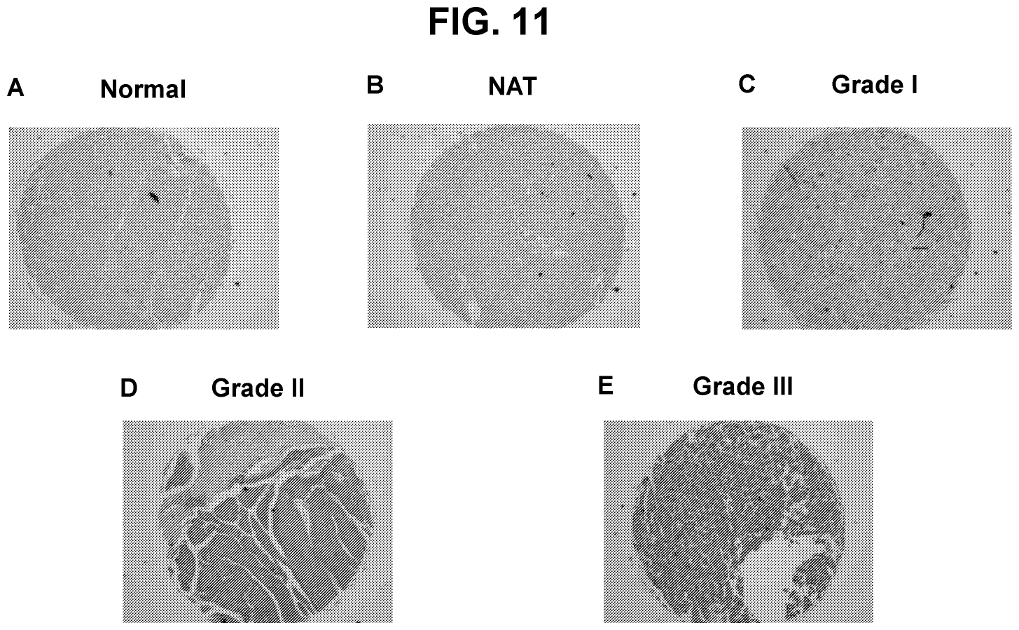

MOSPD2 expression also correlated with malignancy. FIGS. 11A-11E show intense MOSPD2 staining in hepatocellular carcinoma that increased with tumor grade, while normal and NAT samples were negative for MOSPD2 staining.

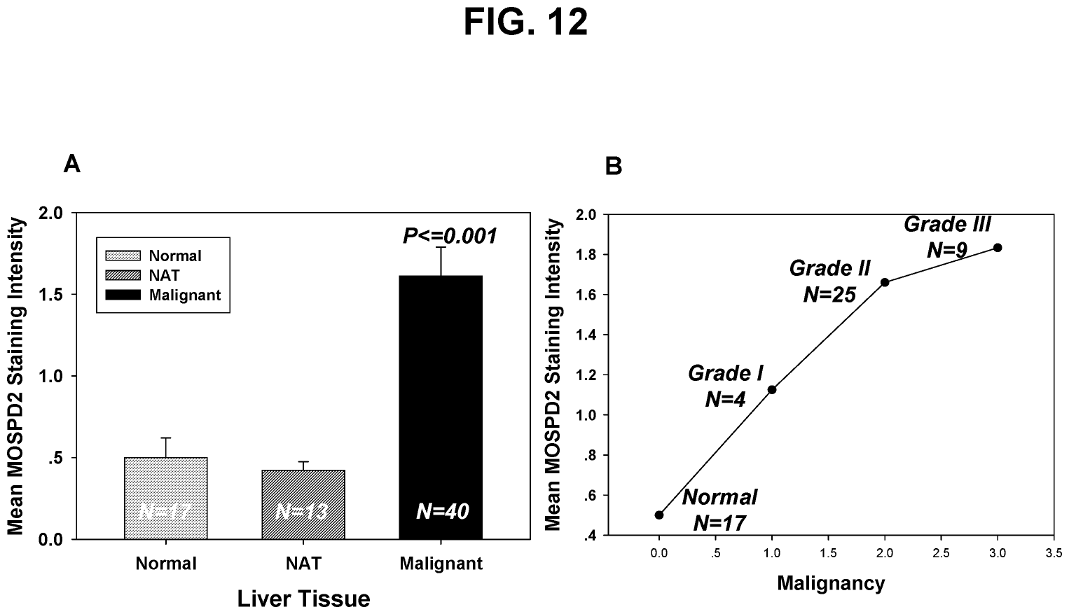

FIGS. 12A-12B summarize the intensity of MOSPD2 staining in malignant liver tissues or controls. MOSPD2 staining intensity was significantly increased by 3.2 or 4 fold in malignant samples in comparison to normal and NAT, respectively (p<0.001). FIG. 12B shows the increase in MOSPD2 staining intensity in different stages of hepatocellular carcinoma.

### Example 11

### Anti-MOSPD2/Anti-CD3 Bispecific Antibody Kills Solid Tumor-Derived Cells and Activates T Cells

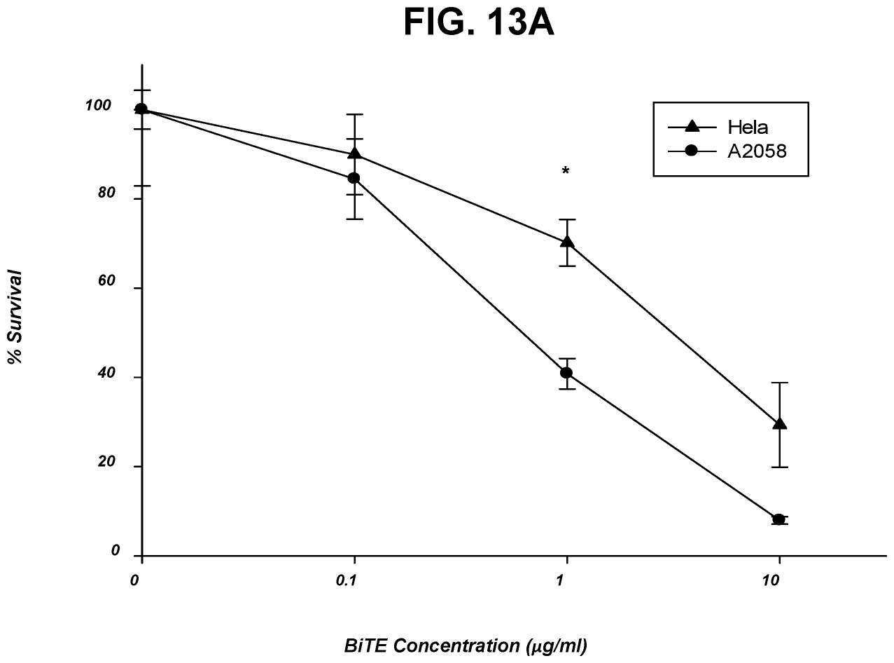

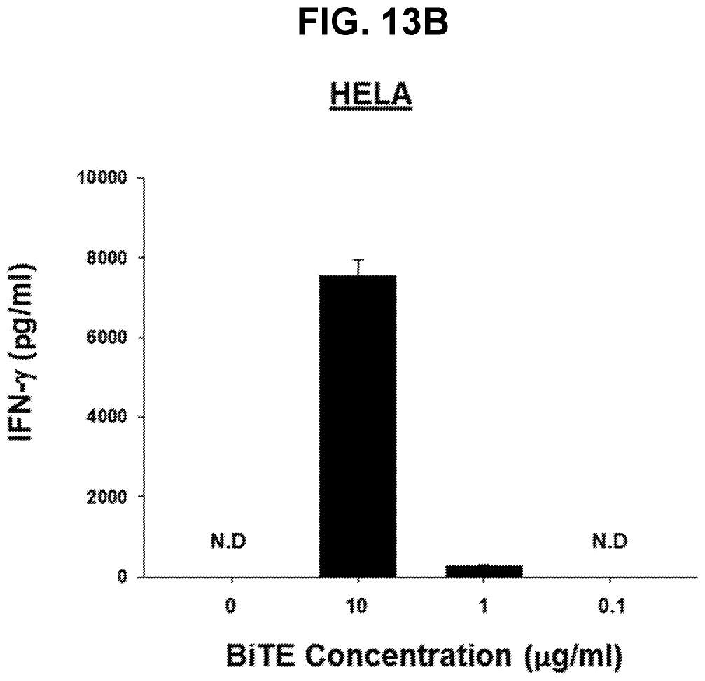

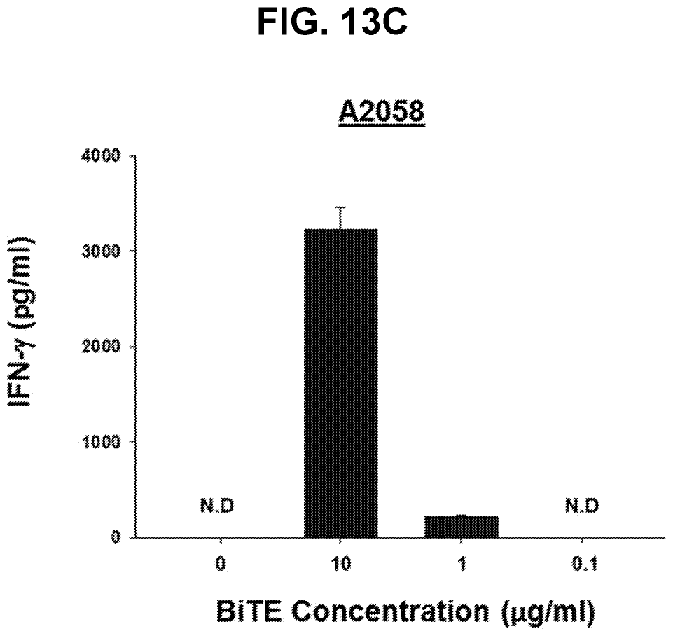

Melanoma (A2058) and cervical (Hela) cancer cell lines were seeded overnight in a 48-well plate (4×10⁴ cells/well) in RPMI-1640 medium containing 10% fetal calf serum (FCS), penicillin and streptomycin. One day later, CD8 effector T-cells primed with a human T cell activation/expansion kit (Miltenyi Biotec, GmbH Cat. No. 130-091-441) were added to the plates at a ratio of 10:1 (Effector cell:T cell) in the presence or absence of anti-MOSPD2/anti-CD3 bispecific antibody (BiTE) at the concentrations indicated in FIG. 13A for 72 hours. The supernatant and cells were then collected, spun at 1,500 rpm for 5 minutes, and kept frozen until tested.

Supernatants were tested by enzyme-linked immunosorbent assay (ELISA) for IFN-γ secretion using the Duoset human IFN-gamma ELISA kit (R&D Systems Cat. No. DY-285). Cancer cells were detached with trypsin-EDTA (0.25%), washed with PBS containing 10% FCS and resuspended in FACS buffer (PBS+2% FCS+0.02% Sodium-azide). Each sample was run by FACS for 2 minutes to assess the number of live cells. Samples were run in triplicate.

FIG. 13A shows that administration of increasing concentrations of anti-MOSPD2/anti-CD3 bispecific antibody (BiTE) results in a dose-dependent decrease in survival of solid tumor-derived cell lines. Results are shown as mean±standard deviation (SD). *=P<0.01.

Supernatants were also analyzed for the secretion of IFN-γ by T cells. Samples were run in duplicate.

FIGS. 13B and 13C show that administration of increasing concentrations of BiTE also results in a dose-dependent increase in IFN-γ release from CD8 effector T cells added to HELA and A2058 cultures, respectively. N.D=not detectable. Results are shown as mean±SD of three samples.

Therefore, in the presence of BiTE, T cells were activated, evidenced by secretion of IFN-γ, and mediated tumor cell death in a dose dependent manner.

### Example 12

### Anti-MOSPD2/Anti-CD3 Bispecific Antibody Kills Myeloid-Derived Cancer Cells and Activates T Cells

THP-1 and U937 myeloid-derived cancer cell lines were seeded overnight in a 48-well plate (4×10⁴ cells/well) in RPMI-1640 medium containing 10% fetal calf serum (FCS), penicillin and streptomycin. T cells were isolated from freshly drawn blood samples using the human pan T cell isolation kit (Miltenyi Biotec, GmbH Cat. No. 130-096-535). T cells were added to the plates at a ratio of 10:1 (Effector cell:T cell) in the presence or absence of 10 μg/ml anti-MOSPD2/anti-CD3 bispecific antibody (BiTE) for 48 hours. The supernatant and cells were then collected, spun at 1,500 rpm for 5 minutes, and kept frozen until tested.

Supernatants were tested by ELISA for IFN-γ secretion using the Duoset human IFN-gamma ELISA kit (R&D Systems Cat. No. DY-285). Cells were stained with anti-human CD3-PE (Thermo Fisher Cat. No. 12-0038) and anti-human CD69-APC (Thermo Fisher Cat. No. 17-0699) in FACS buffer for 30 minutes. Each sample was run by FACS for 2 minutes to assess the number of live cancer cells and the ratio of activated T-cells.

Supernatants were tested by enzyme-linked immunosorbent assay (ELISA) for IFN-γ secretion using the Duoset human IFN-gamma ELISA kit (R&D Systems Cat. No. DY-285). Cancer cells were detached with trypsin-EDTA (0.25%), washed with PBS containing 10% FCS and resuspended in FACS buffer (PBS+2% FCS+0.02% Sodium-azide). Cells were stained with anti-human CD3-PE (Thermo fisher cat #12-0038) and anti-human CD69-APC (Thermo fisher cat #17-0699) in FACS buffer for 30 min. Each sample cells was run by FACS for two minutes to assess the number of live cancer cells and the ratio of activated T-cells. Each sample was run by FACS for 2 minutes to assess the number of live cells. Samples were run in duplicate.

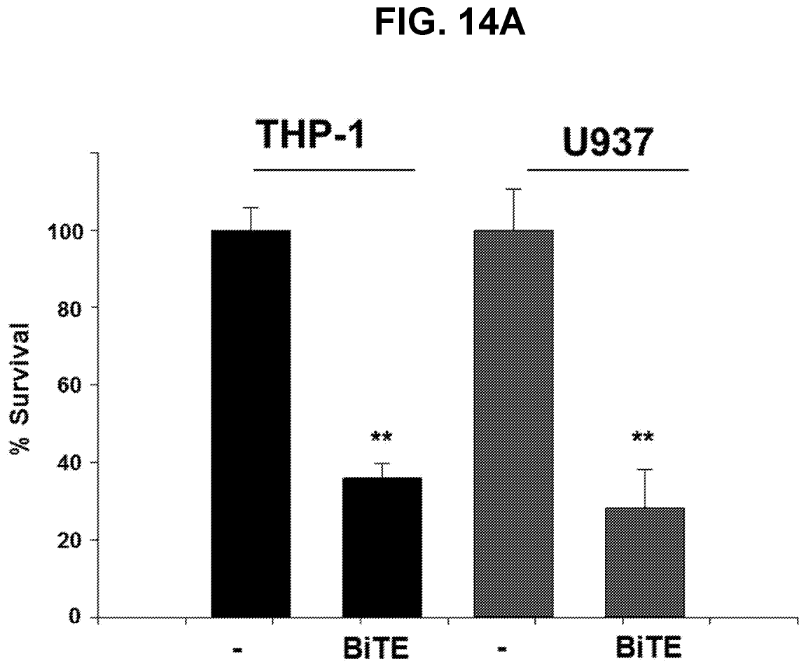

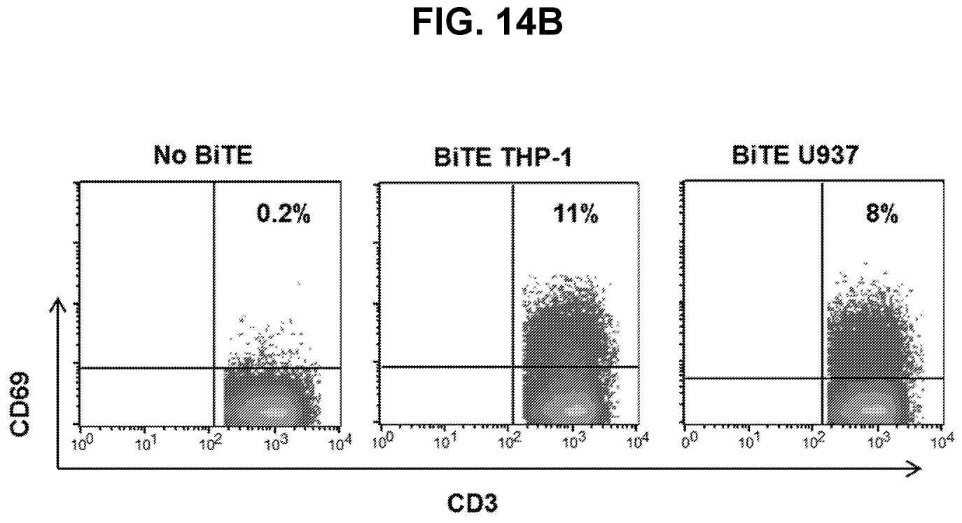

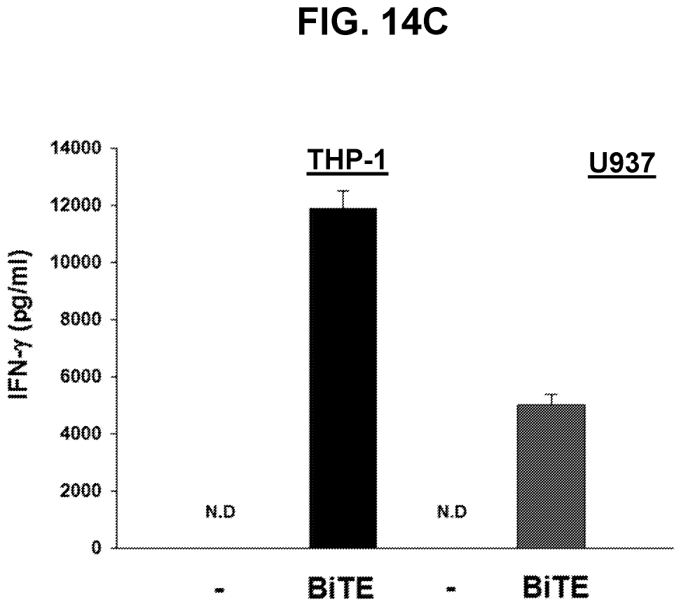

FIG. 14A shows that administration of anti-MOSPD2/anti-CD3 bispecific antibody (BiTE) results in decreased survival of monocytic cell lines. **=P<0.001. FIG. 14B shows staining for CD3 and CD69 of T cells added to THP-1 and U937 cultures. FIG. 14C shows that administration of BiTE also results in an increase in IFN-γ release from T cells added to THP-1 and U937 cultures. N.D=not detectable.

Therefore, in the presence of BiTE, T cells were activated, evidenced by staining for CD3 and CD69 and secretion of IFN-γ, and mediated tumor cell death.

### Example 13

### Anti-MOSPD2/Anti-CD3 Bispecific Antibodies Kill MDA-231 Breast Cancer Cells

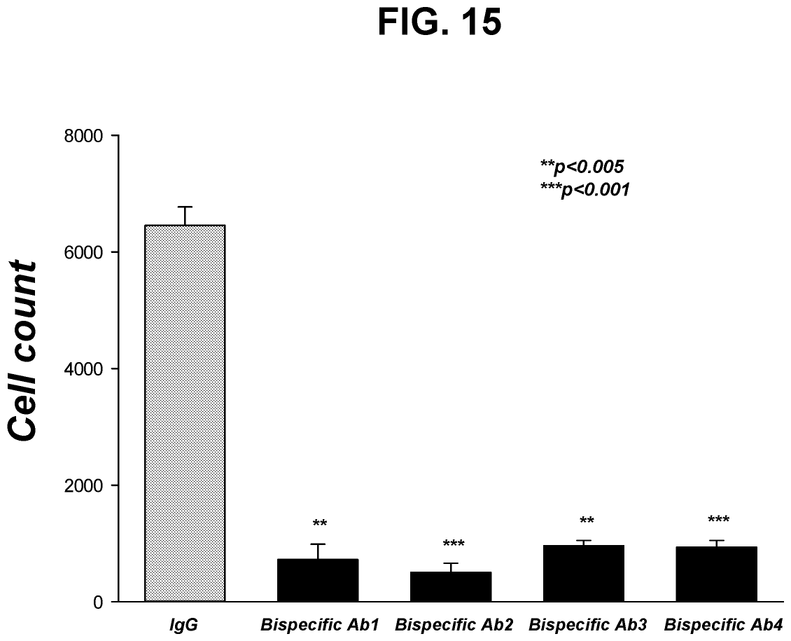

Pre-activated CD8+ T cells were seeded in 48-well plates at a concentration of 2×10⁵ cells/well and co-incubated with MDA-231 breast cancer cells at a concentration of 4×10⁴ cells/well in the presence of 1 μg/ml control IgG antibody or anti-MOSPD2/anti-CD3 bispecific antibodies. After incubation for 24 hours, the cells were washed and treated with trypsin. The cells were then collected and counted using FACScalibur. The resulting cell counts are shown in FIG. 15 as a mean cell count±standard error from triplicate wells. **p<0.005; ***p<0.001. FIG. 15 shows that administration of anti-MOSPD2/anti-CD3 bispecific antibodies results in a significant decrease in survival of breast cancer cells.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A bispecific antibody or antigen binding fragment thereof, comprising (i) one or more antigen binding domains to MOSPD2, and (ii) one or more antigen binding domains to a T cell- or NK cell-specific receptor molecule.

2. The bispecific antibody or antigen binding fragment thereof of claim 1, wherein the T cell- or NK cell-specific receptor molecule is CD3, the T cell receptor (TCR), CD28, CD16, NKG2D, Ox40, 4-1BB, CD2, CD5, or CD95.

3. The bispecific antibody or antigen binding fragment thereof of claim 1 or 2, wherein the one or more antigen binding domains to MOSPD2 is a Fab, Fab′, F(ab′)₂, Fv, scFv, sdFv fragment, heavy chain variable region, light chain variable region, complementarity determining region (CDR), heavy chain CDR1, heavy chain CDR2, heavy chain CDR3, light chain CDR1, light chain CDR2, or light chain CDR3 of an anti-MOSPD2 antibody or antigen binding fragment thereof.

4. The bispecific antibody or antigen binding fragment thereof of any one of claims 1 to 3, wherein the T cell- or NK cell-specific receptor molecule is CD3 and the one or more antigen binding domains to CD3 is a Fab, Fab′, F(ab′)₂, Fv, scFv, sdFv fragment, heavy chain variable region, light chain variable region, CDR, heavy chain CDR1, heavy chain CDR2, heavy chain CDR3, light chain CDR1, light chain CDR2, or light chain CDR3 of an anti-CD3 antibody or antigen binding fragment thereof.

5. The bispecific antibody or antigen binding fragment thereof of any one of claims 1 to 4, comprising one or more of the following antigen binding domains:
(i) a heavy chain variable region of an anti-MOSPD2 antibody or antigen binding fragment thereof; and
(ii) a light chain variable region of an anti-MOSPD2 antibody or antigen binding fragment thereof.

6. The bispecific antibody or antigen binding fragment thereof of any one of claims 1 to 5, comprising one or more of the following antigen binding domains:
(i) a heavy chain variable region of an anti-CD3 antibody or antigen binding fragment thereof; and
(ii) a light chain variable region of an anti-CD3 antibody or antigen binding fragment thereof.

7. The bispecific antibody or antigen binding fragment thereof of any one of claims 1 to 6, comprising the following antigen binding domains:
(i) a heavy chain variable region of an anti-MOSPD2 antibody or antigen binding fragment thereof;
(ii) a light chain variable region of an anti-MOSPD2 antibody or antigen binding fragment thereof;
(iii) a heavy chain variable region of an anti-CD3 antibody or antigen binding fragment thereof; and
(iv) a light chain variable region of an anti-CD3 antibody or antigen binding fragment thereof.

8. The bispecific antibody or antigen binding fragment thereof of any one of claims 1 to 7, wherein one or more of the antigen binding domains are joined by a peptide linker.

9. The bispecific antibody or antigen binding fragment thereof of any one of claims 1 to 8, wherein at least one antigen binding domain is human.

10. The bispecific antibody or antigen binding fragment thereof of any one of claims 1 to 8, wherein at least one antigen binding domain is humanized.

11. The bispecific antibody or antigen binding fragment thereof of any one of claims 1 to 10, wherein the bispecific antibody or antigen binding fragment thereof is a single-chain polypeptide.

12. The bispecific antibody or antigen binding fragment thereof of any one of claims 1 to 11, wherein the bispecific antibody or antigen binding fragment thereof has a molecular weight of no more than about 60,000 Daltons.

13. The bispecific antibody or antigen binding fragment thereof of any one of claims 1 to 12, wherein the bispecific antibody is a nanobody, diabody, duobody, CrossMab, bivalent antibody, bispecific T cell engager (BiTE), dual affinity retargeting (DART), triple body, miniantibody, TriBi minibody, intrabody, or quadroma.

14. The bispecific antibody or antigen binding fragment thereof of any one of claims 1 to 13, wherein the bispecific antibody or antigen binding fragment thereof specifically binds to MOSPD2 and/or CD3 with an equilibrium dissociation constant (K_{D}) of from about 10⁻⁶ M to about 10⁻¹² M.

15. The bispecific antibody or antigen binding fragment thereof of any one of claims 1 to 14, wherein the bispecific antibody or antigen binding fragment, or one or more antigen binding domains to MOSPD2, specifically binds to one or more of SEQ ID NOs:1-4, or a functional variant thereof.

16. The bispecific antibody or antigen binding fragment thereof of any one of claims 1 to 15, wherein the bispecific antibody or antigen binding fragment, or one or more antigen binding domains to MOSPD2, specifically binds to a polypeptide encoded by one or more of SEQ ID NOs:5-8, or a functional variant thereof.

17. The bispecific antibody or antigen binding fragment thereof of any one of claims 1 to 16, wherein the bispecific antibody or antigen binding fragment, or one or more antigen binding domains to MOSPD2, specifically binds to MOSPD2 with a K_{D} of from about 10⁻⁶ M to about 10⁻¹² M.

18. A pharmaceutical composition, comprising the bispecific antibody or antigen binding fragment thereof of any one of claims 1 to 17, and a pharmaceutically acceptable carrier.

19. The pharmaceutical composition of claim 18, suitable for systemic administration.

20. The pharmaceutical composition of claim 18, suitable for local administration.

21. The pharmaceutical composition of claim 18, suitable for oral administration.

22. The pharmaceutical composition of claim 18, suitable for nasal administration.

23. The pharmaceutical composition of claim 18, suitable for intra-peritoneal administration.

24. The pharmaceutical composition of claim 18, suitable for intra-tumor administration.

25. The pharmaceutical composition of claim 18, suitable for intravenous administration.

26. The pharmaceutical composition of claim 18, suitable for intramuscular administration.

27. The pharmaceutical composition of claim 18, suitable for subcutaneous administration.

28. A method of treating or preventing cancer in a subject, comprising administering to the subject the bispecific antibody or antigen binding fragment thereof of any one of claims 1 to 17 or the pharmaceutical composition of any one of claims 18 to 27 in an effective amount to treat or prevent cancer.

29. A method of treating or preventing cancer metastasis in a subject, comprising administering to the subject the bispecific antibody or antigen binding fragment thereof of any one of claims 1 to 17 or the pharmaceutical composition of any one of claims 18 to 27 in an effective amount to treat or prevent cancer metastasis.

30. The method of claim 28 or 29, further comprising administering to the subject an effective amount of an anticancer drug.

31. The method of claim 30, wherein the anticancer drug is Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and I 131 Iodine Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPDX, Carboplatin, Carboplatin-Taxol, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, Chlorambucil-Prednisone, CHOP, Cisplatin, Clafen (Cyclophosphamide), Clofarabine, CMF,Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), Folfiri, Folfiri-Bevacizumab, Folfiri-Cetuximab, Folfirinox, Folflox, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, OEPA, Ofatumumab, OFF, Olaparib, Omacetaxine Mepesuccinate, Oncaspar (Pegaspargase), Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and I 131 Iodine Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), Zydelig (Idelalisib), Zykadia (Ceritinib), or Zytiga (Abiraterone Acetate).

32. A method for inhibiting or reducing tumor cells in a subject, comprising administering to the subject the bispecific antibody or antigen binding fragment thereof of any one of claims 1 to 17 or the pharmaceutical composition of any one of claims 18 to 27.

33. The method of claim 32, wherein the number of tumor cells is reduced by at least about 10%, at least about 20%, at least about 30%, at least about 40%, at least about 50%, at least about 60%, at least about 70%, at least about 80%, at least about 90%, or at least about 100%, compared to a control or reference value.

34. A method of increasing the production of cytokines by cells expressing CD3 in a subject, comprising administering to the subject the bispecific antibody or antigen binding fragment thereof of any one of claims 1 to 17 or the pharmaceutical composition of any one of claims 18 to 27.

35. A method of increasing IL-2, CD69, and/or IFN-γ production or concentration in a T cell, comprising contacting the T cell with the bispecific antibody or antigen binding fragment thereof of any one of claims 1 to 17 or the pharmaceutical composition of any one of claims 18 to 27.

36. The method of claim 35, wherein IFN-γ production increases by at least about 100%, at least about 200%, at least about 300%, at least about 400%, at least about 500%, at least about 600%, at least about 700%, at least about 800%, at least about 900%, or at least about 1000% compared to a control or reference value.

37. The method of claim 35, wherein IFN-γ concentration increases by at least about 1000 pg/ml, at least about 2000 pg/ml, at least about 3000 pg/ml, at least about 4000 pg/ml, at least about 5000 pg/ml, at least about 6000 pg/ml, at least about 7000 pg/ml, at least about 8000 pg/ml, at least about 9000 pg/ml, or at least about 10000 pg/ml compared to a control or reference value.

38. The method of claim 35, wherein CD69 production increases by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, or at least about 30% compared to a control or reference value.

39. A method of stimulating an immune response in a subject, comprising administering to the subject the bispecific antibody or antigen binding fragment thereof of any one of claims 1 to 17 or the pharmaceutical composition of any one of claims 18 to 27.

40. A method of stimulating a T cell-mediated cytotoxic immune response against a cancer cell in a subject, comprising administering to the subject the bispecific antibody or antigen binding fragment thereof of any one of claims 1 to 17 or the pharmaceutical composition of any one of claims 18 to 27.

41. A method of increasing T cell proliferation, comprising contacting a T cell with the bispecific antibody or antigen binding fragment thereof of any one of claims 1 to 17 or the pharmaceutical composition of any one of claims 18 to 27.

42. A method of reducing or depleting the number of T regulatory cells in a tumor of a subject, comprising administering to the subject the bispecific antibody or antigen binding fragment thereof of any one of claims 1 to 17 or the pharmaceutical composition of any one of claims 18 to 27.

43. A method for the prediction, diagnosis, or prognosis of cancer or cancer metastasis in a subject, comprising determining the expression level of MOSPD2 in a sample of the subject using the bispecific antibody or antigen binding fragment thereof of any one of claims 1 to 17.

44. The method of claim 43, comprising (i) determining or quantifying the expression level of MOSPD2 in a sample of the subject using the bispecific antibody or antigen binding fragment thereof of any one of claims 1 to 17, and (ii) comparing the expression level obtained in step (i) with a control or reference value, wherein an increased expression level of MOSPD2 with respect to the control or reference value is indicative of cancer, an increased risk of developing cancer, or a poor cancer prognosis.

45. A method for the prediction, diagnosis, or prognosis of tumor progression or tumor invasiveness in a subject, comprising determining the expression level of MOSPD2 in a sample of the subject using the bispecific antibody or antigen binding fragment thereof of any one of claims 1 to 17.

46. The method of claim 45, comprising (i) determining or quantifying the expression level of MOSPD2 in a sample of the subject using the bispecific antibody or antigen binding fragment thereof of any one of claims 1 to 17, and (ii) comparing the expression level obtained in step (i) with a control or reference value, wherein an increased expression level of MOSPD2 with respect to the control or reference value is indicative or a poor tumor progression or tumor invasiveness prognosis.

47. The method of any one of claims 43 to 46, further comprising one or more of the following steps:
instructing a laboratory to quantify the expression level of MOSPD2 in the sample;
obtaining a report of the expression level of MOSPD2 in the sample from a laboratory; and/or
administering a therapeutically effective amount of an inhibitor of MOSPD2 to the subject.

48. The method of any one of claims 43 to 47, wherein the sample is a tissue biopsy, tumor biopsy, or blood sample from the subject.

49. The method of any one of claims 43 to 48, wherein the control or reference value is the expression level of MOSPD2 in normal tissue or normal adjacent tissue (NAT).

50. The method of any one of claims 43 to 48, wherein the control or reference value is no detectable MOSPD2 expression or no significant MOSPD2 expression.

51. A method for treating or preventing a MOSPD2-expressing tumor in a subject, comprising administering to the subject the bispecific antibody or antigen binding fragment thereof of any one of claims 1 to 17 or the pharmaceutical composition of any one of claims 18 to 27 in an effective amount to treat or prevent a MOSPD2 expressing tumor.

52. A method for treating or preventing a tumor having MOSPD2-expressing tumor associated macrophages in a subject, comprising administering to the subject the bispecific antibody or antigen binding fragment thereof of any one of claims 1 to 17 or the pharmaceutical composition of any one of claims 18 to 27 in an effective amount to treat or prevent a tumor having MOSPD2-expressing tumor associated macrophages.

53. A kit, comprising (i) the bispecific antibody or antigen binding fragment thereof of any one of claims 1 to 17 or the pharmaceutical composition of any one of claims 18 to 27, and (ii) instructions for use.


SEQUENCE LISTING (8 sequences)

SEQ ID NO: 1 - protein, 518 aa, Homo sapiens
  motile sperm domain containing 2, protein variant 1
MAENHAQNKAKLISETRRRFEAEYVTDKSDKYDARDVERLQQDDNWVESYLSWRHNIVDETLKMLDESFQWRKEISVNDLNESSIPRWLLEIGVIYLHGYDKEGNKLFWIRVKYHVKDQKTILDKKKLIAFWLERYAKRENGKPVTVMFDLSETGINSIDMDFVRFIINCFKVYYPKYLSKIVIFDMPWLMNAAFKIVKTWLGPEAVSLLKFTSKNEVQDYVSVEYLPPHMGGTDPFKYSYPPLVDDDFQTPLCENGPITSEDETSSKEDIESDGKETLETISNEEQTPLLKKINPTESTSKAEENEKVDSKVKAFKKPLSVFKGPLLHISPAEELYFGSTESGEKKTLIVLTNVTKNIVAFKVRTTAPEKYRVKPSNSSCDPGASVDIVVSPHGGLTVSAQDRFLIMAAEMEQSSGTGPAELTQFWKEVPRNKVMEHRLRCHTVESSKPNTLTLKDNAFNMSDKTSEDICLQLSRLLESNRKLEDQVQRCIWFQQLLLSLTMLLLAFVTSFFYLLYS

SEQ ID NO: 2 - protein, 455 aa, Homo sapiens
  motile sperm domain containing 2, protein variant 2
MLDESFQWRKEISVNDLNESSIPRWLLEIGVIYLHGYDKEGNKLFWIRVKYHVKDQKTILDKKKLIAFWLERYAKRENGKPVTVMFDLSETGINSIDMDFVRFIINCFKVYYPKYLSKIVIFDMPWLMNAAFKIVKTWLGPEAVSLLKFTSKNEVQDYVSVEYLPPHMGGTDPFKYSYPPLVDDDFQTPLCENGPITSEDETSSKEDIESDGKETLETISNEEQTPLLKKINPTESTSKAEENEKVDSKVKAFKKPLSVFKGPLLHISPAEELYFGSTESGEKKTLIVLTNVTKNIVAFKVRTTAPEKYRVKPSNSSCDPGASVDIVVSPHGGLTVSAQDRFLIMAAEMEQSSGTGPAELTQFWKEVPRNKVMEHRLRCHTVESSKPNTLTLKDNAFNMSDKTSEDICLQLSRLLESNRKLEDQVQRCIWFQQLLLSLTMLLLAFVTSFFYLLYS

SEQ ID NO: 3 - protein, 486 aa, Homo sapiens
  motile sperm domain containing 2, protein variant X1
MAENHAQNKAKLISETRRRFEAEYVTDKSDKYDARDVERLQQDDNWVESYLSWRHNIVDETLKMLDESFQWRKEISVNDLNESSIPRWLLEIGVIYLHGYDKEGNKLFWIRVKYHVKDQKTILDKKKLIAFWLERYAKRENGKPVTVMFDLSETGINSIDMDFVRFIINCFKVYYPKYLSKIVIFDMPWLMNAAFKIVKTWLGPEAVSLLKFTSKNEVQDYVSVEYLPPHMGGTDPFKYSYPPLVDDDFQTPLCENGPITSEDETSSKEDIESDGKETLETISNEEQTPLLKKINPTESTSKAEENEKVDSKVKAFKKPLSVFKGPLLHISPAEELYFGSTESGEKKTLIVLTNVTKNIVAFKVRTTAPEKYRVKPSNSSCDPGASVDIVVSPHGGLTVSAQDRFLIMAAEMEQSSGTGPAELTQFWKEVPRNKVMEHRLRCHTVESSKPNTLTLKDNAFNMSDKTSEDICLQFATSSCEMDCSPP

SEQ ID NO: 4 - protein, 475 aa, Homo sapiens
  motile sperm domain containing 2, protein variant X2
MAENHAQNKAKLISETRRRFEAEYVTDKSDKYDARDVERLQQDDNWVESYLSWRHNIVDETLKMLDESFQWRKEISVNDLNESSIPRWLLEIGVIYLHGYDKEGNKLFWIRVKYHVKDQKTILDKKKLIAFWLERYAKRENGKPVTVMFDLSETGINSIDMDFVRFIINCFKVYYPKYLSKIVIFDMPWLMNAAFKIVKTWLGPEAVSLLKFTSKNEVQDYVSVEYLPPHMGGTDPFKYSYPPLVDDDFQTPLCENGPITSEDETSSKEDIESDGKETLETISNEEQTPLLKKINPTESTSKAEENEKVDSKVKAFKKPLSVFKGPLLHISPAEELYFGSTESGEKKTLIVLTNVTKNIVAFKVRTTAPEKYRVKPSNSSCDPGASVDIVVSPHGGLTVSAQDRFLIMAAEMEQSSGTGPAELTQFWKEVPRNKVMEHRLRCHTVESSKPNTLTLKDNAFNMSDKTSEDICLQYS

SEQ ID NO: 5 - DNA, 3219 bp, Homo sapiens
  motile sperm domain containing 2, transcript variant 1, mRNA, coding region 125-1678
accgcctccccctcccacccttctctgtctacctctgggcgggactgccgggtgatgagatactcggtcggcgacggtagaacgggcgacggcgacaaccgcaatcacatccacgacggtgatcatggcagagaatcacgcccagaataaagccaagctcatctctgagacccggaggaggttcgaagctgagtatgtgacagataagtcagataaatatgatgcacgtgatgttgaaaggctacaacaagatgataactgggttgaaagttacttatcttggagacataatattgtagatgaaacactgaagatgctcgatgagagttttcagtggaggaaagaaatttctgtcaatgaccttaatgaatcctccattcccagatggttattggaaattggtgttatttatctccatggttatgacaaagaaggtaacaaattgttctggatcagggtgaagtatcatgtaaaagaccagaaaaccatattggacaaaaagaagctcatagcattctggttggaacgttatgctaagagggaaaatgggaaacctgtaacagtgatgtttgacctgtcagaaactggaataaatagcattgacatggactttgtacgctttatcatcaactgctttaaggtttattaccctaaatacctctcaaaaatagtgatctttgatatgccttggttaatgaatgctgctttcaaaattgtgaaaacctggcttggtccagaagcagtgagcttgttgaagtttacaagcaaaaatgaagtccaggactatgtcagtgtagaatacctgcctccccacatgggtggaactgatcctttcaagtatagctatccaccactagtagatgatgacttccagaccccactgtgtgagaatgggcctattaccagtgaggatgaaacttcaagtaaagaagacatagaaagtgatggcaaagaaacattggaaacaatttctaatgaagaacaaacacctcttcttaaaaagattaacccaaccgaatctacttccaaagcagaagaaaatgaaaaagttgattcaaaagtgaaagctttcaagaaaccattgagtgtatttaaaggccccttactacacatcagcccagcagaagaactgtactttggaagtacagaatccggagagaagaaaaccttaatagtgttgacaaatgtaactaaaaatatagtggcatttaaggtgagaacaacagctccagaaaaatacagagtcaagccaagcaatagcagctgtgacccgggtgcatcagtggatatagttgtgtctccccatgggggtttaacagtctctgcccaagaccgttttctgataatggctgcagaaatggaacagtcatctggcacaggcccagcagaattaactcagttttggaaagaagttcccagaaacaaagtgatggaacataggttaagatgccatactgttgaaagcagtaaaccaaacactcttacgttaaaagacaatgctttcaatatgtcagataaaaccagtgaagatatatgtctacaactcagtcgtttactagaaagcaataggaagcttgaagaccaagttcagcgttgtatctggttccagcagctgctgctttccttaacaatgctcttgcttgcttttgtcacctctttcttctatttattgtacagttaaagaagtggtgccgggtaggaaccacggttccttcgtccattagttggaaaaagtaacagacctaaaactctaccaagctactaaaaacattgcacatctgtgcttcctaaaaggaaatatgcagcacgtggaggggaacacatacatgtcttgaaaataaactgctagaataaagaaatgctggagaaattgattataagagactatagctatttagtaaagtaagtaaaggcatatccattgtgtaaattaatagtttaaatataatttattttttccttttgatctgaatacttttaaagcttaagttttatcgtgtaaatacattagctaaactgaaaagtataagtaacatgctttgttgcagccaaaaaatgtaatctgcttttttatgacagaattattatagctgagctgacttactagcttttctatactatgtatatagaagaacatgtatattgagaaagaaaacatacttatatagaggaatttatgtaaccatgactttgtaattttgagaattcctcccagtgatggtcagtattcttttggaatgtaaaccgatttaatgccaaaccaccttaacctttgtttctcagtgttccttaacagcctgccttttattaatctcaggcttttttatgaacactctcatttcagtagaatttggaaaactaagcgtggttggaatttctttgaattctgttagtaatgcccaaaagaaaagtctcaagcagtccccctatccagtcatttttatggagtttcatgttgtccactatagctggacactgaaccttttgcctaatttattataaaggcctgaccctctattgtcccatcttcacccccattccagagcagaggagtctctgtggaccatgaattgcactgtctccctcctcatttctaaatgaaaggtattagatataaatttttttgaaaggttagttgtttgagatgctaagcaggataataaatttagattttaaaatgttccctgtaaaagtcagcccatgacaaggaaatttacaaaatactagagtatctagaagggtgaaaacaaaaaaaaataaaaagaaacacagacgcccaggtgtcagctctccgtttaaagaatgaaaaatgtaactcatgatgatctgtgaaaccttcaaactaggaccaattgacttacttgatattctgcctttgatatggtagtacccacccggtattcctaaaatcctaaaaagatacaccttgcagtagcagaggcaatgacatgagtttgttttctcattaatatgaccagtttgggtctatgttggttcacatgtacatctactttatatgaaagaaaaaacagttgtctgcctgtaaaatgttgagtttcgattgagccatgtttggagattttattactattctgaagggtagtgttgttggttttcatcttcaagaagttgattccaaaactgagttatgaagaatgatataacagttccttcaaaattggcctaggaaataaaaccttaaaaggacaaaaaaaaaaa

SEQ ID NO: 6 - DNA, 3183 bp, Homo sapiens
  motile sperm domain containing 2, transcript variant 2, mRNA, coding region 278-1642
agtcacaataataggtacttaaaaacatgtcatatgataggaaactagaataacacacctctaaataatccatggattaccaacttctcagcaatggtgtaaattcttctgctaaataacccgtgggttaaagaggacatcacattggaaattccagagtacttaaaataagtcagataaatatgatgcacgtgatgttgaaaggctacaacaagatgataactgggttgaaagttacttatcttggagacataatattgtagatgaaacactgaagatgctcgatgagagttttcagtggaggaaagaaatttctgtcaatgaccttaatgaatcctccattcccagatggttattggaaattggtgttatttatctccatggttatgacaaagaaggtaacaaattgttctggatcagggtgaagtatcatgtaaaagaccagaaaaccatattggacaaaaagaagctcatagcattctggttggaacgttatgctaagagggaaaatgggaaacctgtaacagtgatgtttgacctgtcagaaactggaataaatagcattgacatggactttgtacgctttatcatcaactgctttaaggtttattaccctaaatacctctcaaaaatagtgatctttgatatgccttggttaatgaatgctgctttcaaaattgtgaaaacctggcttggtccagaagcagtgagcttgttgaagtttacaagcaaaaatgaagtccaggactatgtcagtgtagaatacctgcctccccacatgggtggaactgatcctttcaagtatagctatccaccactagtagatgatgacttccagaccccactgtgtgagaatgggcctattaccagtgaggatgaaacttcaagtaaagaagacatagaaagtgatggcaaagaaacattggaaacaatttctaatgaagaacaaacacctcttcttaaaaagattaacccaaccgaatctacttccaaagcagaagaaaatgaaaaagttgattcaaaagtgaaagctttcaagaaaccattgagtgtatttaaaggccccttactacacatcagcccagcagaagaactgtactttggaagtacagaatccggagagaagaaaaccttaatagtgttgacaaatgtaactaaaaatatagtggcatttaaggtgagaacaacagctccagaaaaatacagagtcaagccaagcaatagcagctgtgacccgggtgcatcagtggatatagttgtgtctccccatgggggtttaacagtctctgcccaagaccgttttctgataatggctgcagaaatggaacagtcatctggcacaggcccagcagaattaactcagttttggaaagaagttcccagaaacaaagtgatggaacataggttaagatgccatactgttgaaagcagtaaaccaaacactcttacgttaaaagacaatgctttcaatatgtcagataaaaccagtgaagatatatgtctacaactcagtcgtttactagaaagcaataggaagcttgaagaccaagttcagcgttgtatctggttccagcagctgctgctttccttaacaatgctcttgcttgcttttgtcacctctttcttctatttattgtacagttaaagaagtggtgccgggtaggaaccacggttccttcgtccattagttggaaaaagtaacagacctaaaactctaccaagctactaaaaacattgcacatctgtgcttcctaaaaggaaatatgcagcacgtggaggggaacacatacatgtcttgaaaataaactgctagaataaagaaatgctggagaaattgattataagagactatagctatttagtaaagtaagtaaaggcatatccattgtgtaaattaatagtttaaatataatttattttttccttttgatctgaatacttttaaagcttaagttttatcgtgtaaatacattagctaaactgaaaagtataagtaacatgctttgttgcagccaaaaaatgtaatctgcttttttatgacagaattattatagctgagctgacttactagcttttctatactatgtatatagaagaacatgtatattgagaaagaaaacatacttatatagaggaatttatgtaaccatgactttgtaattttgagaattcctcccagtgatggtcagtattcttttggaatgtaaaccgatttaatgccaaaccaccttaacctttgtttctcagtgttccttaacagcctgccttttattaatctcaggcttttttatgaacactctcatttcagtagaatttggaaaactaagcgtggttggaatttctttgaattctgttagtaatgcccaaaagaaaagtctcaagcagtccccctatccagtcatttttatggagtttcatgttgtccactatagctggacactgaaccttttgcctaatttattataaaggcctgaccctctattgtcccatcttcacccccattccagagcagaggagtctctgtggaccatgaattgcactgtctccctcctcatttctaaatgaaaggtattagatataaatttttttgaaaggttagttgtttgagatgctaagcaggataataaatttagattttaaaatgttccctgtaaaagtcagcccatgacaaggaaatttacaaaatactagagtatctagaagggtgaaaacaaaaaaaaataaaaagaaacacagacgcccaggtgtcagctctccgtttaaagaatgaaaaatgtaactcatgatgatctgtgaaaccttcaaactaggaccaattgacttacttgatattctgcctttgatatggtagtacccacccggtattcctaaaatcctaaaaagatacaccttgcagtagcagaggcaatgacatgagtttgttttctcattaatatgaccagtttgggtctatgttggttcacatgtacatctactttatatgaaagaaaaaacagttgtctgcctgtaaaatgttgagtttcgattgagccatgtttggagattttattactattctgaagggtagtgttgttggttttcatcttcaagaagttgattccaaaactgagttatgaagaatgatataacagttccttcaaaattggcctaggaaataaaaccttaaaaggacaaaaaaaaaaa

SEQ ID NO: 7 - DNA, 2330 bp, Homo sapiens
  motile sperm domain containing 2, transcript variant X1, mRNA, coding region 125-1582
accgcctccccctcccacccttctctgtctacctctgggcgggactgccgggtgatgagatactcggtcggcgacggtagaacgggcgacggcgacaaccgcaatcacatccacgacggtgatcatggcagagaatcacgcccagaataaagccaagctcatctctgagacccggaggaggttcgaagctgagtatgtgacagataagtcagataaatatgatgcacgtgatgttgaaaggctacaacaagatgataactgggttgaaagttacttatcttggagacataatattgtagatgaaacactgaagatgctcgatgagagttttcagtggaggaaagaaatttctgtcaatgaccttaatgaatcctccattcccagatggttattggaaattggtgttatttatctccatggttatgacaaagaaggtaacaaattgttctggatcagggtgaagtatcatgtaaaagaccagaaaaccatattggacaaaaagaagctcatagcattctggttggaacgttatgctaagagggaaaatgggaaacctgtaacagtgatgtttgacctgtcagaaactggaataaatagcattgacatggactttgtacgctttatcatcaactgctttaaggtttattaccctaaatacctctcaaaaatagtgatctttgatatgccttggttaatgaatgctgctttcaaaattgtgaaaacctggcttggtccagaagcagtgagcttgttgaagtttacaagcaaaaatgaagtccaggactatgtcagtgtagaatacctgcctccccacatgggtggaactgatcctttcaagtatagctatccaccactagtagatgatgacttccagaccccactgtgtgagaatgggcctattaccagtgaggatgaaacttcaagtaaagaagacatagaaagtgatggcaaagaaacattggaaacaatttctaatgaagaacaaacacctcttcttaaaaagattaacccaaccgaatctacttccaaagcagaagaaaatgaaaaagttgattcaaaagtgaaagctttcaagaaaccattgagtgtatttaaaggccccttactacacatcagcccagcagaagaactgtactttggaagtacagaatccggagagaagaaaaccttaatagtgttgacaaatgtaactaaaaatatagtggcatttaaggtgagaacaacagctccagaaaaatacagagtcaagccaagcaatagcagctgtgacccgggtgcatcagtggatatagttgtgtctccccatgggggtttaacagtctctgcccaagaccgttttctgataatggctgcagaaatggaacagtcatctggcacaggcccagcagaattaactcagttttggaaagaagttcccagaaacaaagtgatggaacataggttaagatgccatactgttgaaagcagtaaaccaaacactcttacgttaaaagacaatgctttcaatatgtcagataaaaccagtgaagatatatgtctacaatttgccacctccagctgtgaaatggactgcagtccaccctaagtactgtgcacagtatctccctgtgtgtgtgcacagtggcttccccttacatggtagatttttggccttaatataatctaatcccaaagtagttgtgtatgttttctgttccttggcaaataaatgaagaaataattagccaagattgaaaatgtattgtcctaacggtgtccctttaatgtttcatatgaaaaattatgttgacccactaaaatatccttgctcaatgtctggtcagttgaatttaataacatatcttgttaatgtttgtgtgtctattaaatgtgactaagcaggattactgaaaattcactataaaatcaaaggcatctaaacgtttgtacttgtcttgattaatcatatatttacacttgatttttttctgtcttcatttgtttttatttaatcataattgcatgatttttttggtactctaatcagtaattttatttttaatcatgtcattacctattcatgaccaaattaccaaggaaccaacatttagatttagatatttgttttcacttaggaatggaaattaatagattttccatgaaagcattagtgaaatatcattaccttgatctgcaagtagcctaaaaatgcgattgctggtaaacctggcctcaaatttcatactaccataactgtttttatatattgccactaattttgactggatttaatagcactttattgtacaactacaaaaaaaaatatattcctagaattgttgccagtgtaa

SEQ ID NO: 8 - DNA, 3909 bp, Homo sapiens
  motile sperm domain containing 2, transcript variant X2, mRNA, coding region 125-1549
accgcctccccctcccacccttctctgtctacctctgggcgggactgccgggtgatgagatactcggtcggcgacggtagaacgggcgacggcgacaaccgcaatcacatccacgacggtgatcatggcagagaatcacgcccagaataaagccaagctcatctctgagacccggaggaggttcgaagctgagtatgtgacagataagtcagataaatatgatgcacgtgatgttgaaaggctacaacaagatgataactgggttgaaagttacttatcttggagacataatattgtagatgaaacactgaagatgctcgatgagagttttcagtggaggaaagaaatttctgtcaatgaccttaatgaatcctccattcccagatggttattggaaattggtgttatttatctccatggttatgacaaagaaggtaacaaattgttctggatcagggtgaagtatcatgtaaaagaccagaaaaccatattggacaaaaagaagctcatagcattctggttggaacgttatgctaagagggaaaatgggaaacctgtaacagtgatgtttgacctgtcagaaactggaataaatagcattgacatggactttgtacgctttatcatcaactgctttaaggtttattaccctaaatacctctcaaaaatagtgatctttgatatgccttggttaatgaatgctgctttcaaaattgtgaaaacctggcttggtccagaagcagtgagcttgttgaagtttacaagcaaaaatgaagtccaggactatgtcagtgtagaatacctgcctccccacatgggtggaactgatcctttcaagtatagctatccaccactagtagatgatgacttccagaccccactgtgtgagaatgggcctattaccagtgaggatgaaacttcaagtaaagaagacatagaaagtgatggcaaagaaacattggaaacaatttctaatgaagaacaaacacctcttcttaaaaagattaacccaaccgaatctacttccaaagcagaagaaaatgaaaaagttgattcaaaagtgaaagctttcaagaaaccattgagtgtatttaaaggccccttactacacatcagcccagcagaagaactgtactttggaagtacagaatccggagagaagaaaaccttaatagtgttgacaaatgtaactaaaaatatagtggcatttaaggtgagaacaacagctccagaaaaatacagagtcaagccaagcaatagcagctgtgacccgggtgcatcagtggatatagttgtgtctccccatgggggtttaacagtctctgcccaagaccgttttctgataatggctgcagaaatggaacagtcatctggcacaggcccagcagaattaactcagttttggaaagaagttcccagaaacaaagtgatggaacataggttaagatgccatactgttgaaagcagtaaaccaaacactcttacgttaaaagacaatgctttcaatatgtcagataaaaccagtgaagatatatgtctacaatacagttaaagaagtggtgccgggtaggaaccacggttccttcgtccattagttggaaaaagtaacagacctaaaactctaccaagctactaaaaacattgcacatctgtgcttcctaaaaggaaatatgcagcacgtggaggggaacacatacatgtcttgaaaataaactgctagaataaagaaatgctggagaaattgattataagagactatagctatttagtaaagtaagtaaaggcatatccattgtgtaaattaatagtttaaatataatttattttttccttttgatctgaatacttttaaagcttaagttttatcgtgtaaatacattagctaaactgaaaagtataagtaacatgctttgttgcagccaaaaaatgtaatctgcttttttatgacagaattattatagctgagctgacttactagcttttctatactatgtatatagaagaacatgtatattgagaaagaaaacatacttatatagaggaatttatgtaaccatgactttgtaattttgagaattcctcccagtgatggtcagtattcttttggaatgtaaaccgatttaatgccaaaccaccttaacctttgtttctcagtgttccttaacagcctgccttttattaatctcaggcttttttatgaacactctcatttcagtagaatttggaaaactaagcgtggttggaatttctttgaattctgttagtaatgcccaaaagaaaagtctcaagcagtccccctatccagtcatttttatggagtttcatgttgtccactatagctggacactgaaccttttgcctaatttattataaaggcctgaccctctattgtcccatcttcacccccattccagagcagaggagtctctgtggaccatgaattgcactgtctccctcctcatttctaaatgaaaggtattagatataaatttttttgaaaggttagttgtttgagatgctaagcaggataataaatttagattttaaaatgttccctgtaaaagtcagcccatgacaaggaaatttacaaaatactagagtatctagaagggtgaaaacaaaaaaaaataaaaagaaacacagacgcccaggtgtcagctctccgtttaaagaatgaaaaatgtaactcatgatgatctgtgaaaccttcaaactaggaccaattgacttacttgatattctgcctttgatatggtagtacccacccggtattcctaaaatcctaaaaagatacaccttgcagtagcagaggcaatgacatgagtttgttttctcattaatatgaccagtttgggtctatgttggttcacatgtacatctactttatatgaaagaaaaaacagttgtctgcctgtaaaatgttgagtttcgattgagccatgtttggagattttattactattctgaagggtagtgttgttggttttcatcttcaagaagttgattccaaaactgagttatgaagaatgatataacagttccttcaaaattggcctaggaaataaaaccttaaaaggacactggtgtgctactttgtcttaatttgggcttttctgtttcagtttgccacctccagctgtgaaatggactgcagtccaccctaagtactgtgcacagtatctccctgtgtgtgtgcacagtggcttccccttacatggtagatttttggccttaatataatctaatcccaaagtagttgtgtatgttttctgttccttggcaaataaatgaagaaataattagccaagattgaaaatgtattgtcctaacggtgtccctttaatgtttcatatgaaaaattatgttgacccactaaaatatccttgctcaatgtctggtcagttgaatttaataacatatcttgttaatgtttgtgtgtctattaaatgtgactaagcaggattactgaaaattcactataaaatcaaaggcatctaaacgtttgtacttgtcttgattaatcatatatttacacttgatttttttctgtcttcatttgtttttatttaatcataattgcatgatttttttggtactctaatcagtaattttatttttaatcatgtcattacctattcatgaccaaattaccaaggaaccaacatttagatttagatatttgttttcacttaggaatggaaattaatagattttccatgaaagcattagtgaaatatcattaccttgatctgcaagtagcctaaaaatgcgattgctggtaaacctggcctcaaatttcatactaccataactgtttttatatattgccactaattttgactggatttaatagcactttattgtacaactacaaaaaaaaatatattcctagaattgttgccagtgtaa